SEQUENCE LISTING (31 sequences)

SEQ ID NO: 1 - DNA, 336 bp, synthetic construct
  source 1..336: mol_type = other DNA
gaggtgcagctgcagcagtctggcgctgaactcgtgcggcctggcgctctcgtgaagctgagctgtaaagccagcggcctgaacatcaaggactactacatgcactgggtcaagcagcggcccgagcagggcctggaatggatcggctggatcgaccccgagaacgacaacaccagattcgacccccggttccagggccgggtgtccatcattgccgacaccagcagcaacaccgcctacctgcagctgtccagcctgaccagcgaggacaccgccgtgtactattgtgccgccgaggcctattggggccagggcacactcgtgacagtgtctgct

SEQ ID NO: 2 - DNA, 330 bp, synthetic construct
  source 1..330: mol_type = other DNA
gatatcgtgctgacacagagccccgcctccctggatgtgtccctgggacagagagccacaatcagctgcagagcctcccagagcgtgtccaccagctcctacagctatatgcactggtatcagcagaagcccggccagagccccaagctgctgattaagtacgccagcaacctggaaagcggcgtgcccgccagattttctggcagcggctctggcaccgacttcaccctgaatatccaccccgtggaagaggaagataccgccacctactactgccagcacagctgggagatctggaccttcggcggaggcaccaagctggaaatcaag

SEQ ID NO: 3 - protein, 112 aa, synthetic construct
EVQLQQSGAELVRPGALVKLSCKASGLNIKDYYMHWVKQRPEQGLEWIGWIDPENDNTRFDPRFQGRVSIIADTSSNTAYLQLSSLTSEDTAVYYCAAEAYWGQGTLVTVSA

SEQ ID NO: 4 - protein, 110 aa, synthetic construct
DIVLTQSPASLDVSLGQRATISCRASQSVSTSSYSYMHWYQQKPGQSPKLLIKYASNLESGVPARFSGSGSGTDFTLNIHPVEEEDTATYYCQHSWEIWTFGGGTKLEIK

SEQ ID NO: 5 - protein, 5 aa, synthetic construct
AAEAY

SEQ ID NO: 6 - protein, 8 aa, synthetic construct
IDPENDNT

SEQ ID NO: 7 - protein, 8 aa, synthetic construct
GLNIKDYY

SEQ ID NO: 8 - protein, 8 aa, synthetic construct
QHSWEIWT

SEQ ID NO: 9 - protein, 41 aa, synthetic construct
RSKRSRLLHSDYMNMTPRRPGPTRKHYQPYAPPRDFAAYRS

SEQ ID NO: 10 - protein, 10 aa, synthetic construct
QSVSTSSYSY

SEQ ID NO: 11 - DNA, 2400 bp, synthetic construct
  source 1..2400: mol_type = other DNA
atggccctgcctgtgacagccctgctgctgcctctggctctgctgctgcatgccgccagacctggatctgaggtgcagctgcagcagtctggcgctgaactcgtgcggcctggcgctctcgtgaagctgagctgtaaagccagcggcctgaacatcaaggactactacatgcactgggtcaagcagcggcccgagcagggcctggaatggatcggctggatcgaccccgagaacgacaacaccagattcgacccccggttccagggccgggtgtccatcattgccgacaccagcagcaacaccgcctacctgcagctgtccagcctgaccagcgaggacaccgccgtgtactattgtgccgccgaggcctattggggccagggcacactcgtgacagtgtctgctggcggcggaggatctggcggaggcggaagtggcgggggaggaagcggagggggcggatctgatatcgtgctgacacagagccccgcctccctggatgtgtccctgggacagagagccacaatcagctgcagagcctcccagagcgtgtccaccagctcctacagctatatgcactggtatcagcagaagcccggccagagccccaagctgctgattaagtacgccagcaacctggaaagcggcgtgcccgccagattttctggcagcggctctggcaccgacttcaccctgaatatccaccccgtggaagaggaagataccgccacctactactgccagcacagctgggagatctggaccttcggcggaggcaccaagctggaaatcaagtccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatcatctacatctgggcgcccttggccgggacttgtggggtccttctcctgtcactggttatcaccctttactgcagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgttaacggaagcggcgccaccaacttcagcctgctgaaacaggccggcgacgtggaagagaaccctggccccactagtgccaccatgctcgtgcggagaggcgccagagctggccccagaatgcctagaggatggaccgccctgtgcctgctgagcctgctgcctagcggcttcatgagcctggacaacaacggcaccgccacccctgagctgcctacccagggcaccttcagcaacgtgtccaccaatgtgtcctaccaggaaaccaccacccccagcaccctgggcagcacatctctgcaccctgtgtcccagcacggcaacgaggccaccaccaacatcaccgagacaaccgtgaagttcaccagcaccagcgtgatcacctccgtgtacggcaacaccaacagcagcgtgcagagccagacctccgtgatcagcaccgtgtttaccacccccgctaatgtgtccacccccgaaaccaccctgaagcccagcctgtctcccggaaacgtgtccgacctgagcaccacctctaccagcctggccaccagccccaccaagccttacacaagcagcagccccatcctgagcgacatcaaggccgagatcaagtgcagcggcatccgggaagtgaagctgacacagggcatctgcctggaacagaacaagaccagcagctgcgccgagttcaagaaggacagaggcgagggcctggccagagtgctgtgtggcgaagaacaggccgatgccgatgctggcgctcaagtgtgctctctgctgctggcccagagcgaagtgcggcctcagtgcctgctgctggtgctggccaacagaaccgagatcagcagcaaactgcagctgatgaagaagcaccagagcgacctgaagaagctgggcatcctggacttcaccgagcaggacgtggcctcccaccagagctacagccagaaaaccctgatcgccctcgtgaccagcggagccctgctggcagtgctgggaatcaccggctactttctgatgaaccggcggagctggtcccccaccggcgaaaga

SEQ ID NO: 12 - protein, 799 aa, synthetic construct
MALPVTALLLPLALLLHAARPGSEVQLQQSGAELVRPGALVKLSCKASGLNIKDYYMHWVKQRPEQGLEWIGWIDPENDNTRFDPRFQGRVSIIADTSSNTAYLQLSSLTSEDTAVYYCAAEAYWGQGTLVTVSAGGGGSGGGGSGGGGSGGGGSDIVLTQSPASLDVSLGQRATISCRASQSVSTSSYSYMHWYQQKPGQSPKLLIKYASNLESGVPARFSGSGSGTDFTLNIHPVEEEDTATYYCQHSWEIWTFGGGTKLEIKSGTTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRVNGSGATNFSLLKQAGDVEENPGPTSATMLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGER

SEQ ID NO: 13 - DNA, 2538 bp, synthetic construct
  source 1..2538: mol_type = other DNA
gtggccctgcctgtgacagccctgctgctgcctctggctctgctgctgcatgccgccagacctggatctgaggtgcagctgcagcagtctggcgctgaactcgtgcggcctggcgctctcgtgaagctgagctgtaaagccagcggcctgaacatcaaggactactacatgcactgggtcaagcagcggcccgagcagggcctggaatggatcggctggatcgaccccgagaacgacaacaccagattcgacccccggttccagggccgggtgtccatcattgccgacaccagcagcaacaccgcctacctgcagctgtccagcctgaccagcgaggacaccgccgtgtactattgtgccgccgaggcctattggggccagggcacactcgtgacagtgtctgctggcggcggaggatctggcggaggcggaagtggcgggggaggaagcggagggggcggatctgatatcgtgctgacacagagccccgcctccctggatgtgtccctgggacagagagccacaatcagctgcagagcctcccagagcgtgtccaccagctcctacagctatatgcactggtatcagcagaagcccggccagagccccaagctgctgattaagtacgccagcaacctggaaagcggcgtgcccgccagattttctggcagcggctctggcaccgacttcaccctgaatatccaccccgtggaagaggaagataccgccacctactactgccagcacagctgggagatctggaccttcggcggaggcaccaagctggaaatcaagtccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatcttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgttaacggaagcggcgccaccaacttcagcctgctgaaacaggccggcgacgtggaagagaaccctggccccactagtgccaccatgctcgtgcggagaggcgccagagctggccccagaatgcctagaggatggaccgccctgtgcctgctgagcctgctgcctagcggcttcatgagcctggacaacaacggcaccgccacccctgagctgcctacccagggcaccttcagcaacgtgtccaccaatgtgtcctaccaggaaaccaccacccccagcaccctgggcagcacatctctgcaccctgtgtcccagcacggcaacgaggccaccaccaacatcaccgagacaaccgtgaagttcaccagcaccagcgtgatcacctccgtgtacggcaacaccaacagcagcgtgcagagccagacctccgtgatcagcaccgtgtttaccacccccgctaatgtgtccacccccgaaaccaccctgaagcccagcctgtctcccggaaacgtgtccgacctgagcaccacctctaccagcctggccaccagccccaccaagccttacacaagcagcagccccatcctgagcgacatcaaggccgagatcaagtgcagcggcatccgggaagtgaagctgacacagggcatctgcctggaacagaacaagaccagcagctgcgccgagttcaagaaggacagaggcgagggcctggccagagtgctgtgtggcgaagaacaggccgatgccgatgctggcgctcaagtgtgctctctgctgctggcccagagcgaagtgcggcctcagtgcctgctgctggtgctggccaacagaaccgagatcagcagcaaactgcagctgatgaagaagcaccagagcgacctgaagaagctgggcatcctggacttcaccgagcaggacgtggcctcccaccagagctacagccagaaaaccctgatcgccctcgtgaccagcggagccctgctggcagtgctgggaatcaccggctactttctgatgaaccggcggagctggtcccccaccggcgaaaga

SEQ ID NO: 14 - protein, 846 aa, synthetic construct
MALPVTALLLPLALLLHAARPGSEVQLQQSGAELVRPGALVKLSCKASGLNIKDYYMHWVKQRPEQGLEWIGWIDPENDNTRFDPRFQGRVSIIADTSSNTAYLQLSSLTSEDTAVYYCAAEAYWGQGTLVTVSAGGGGSGGGGSGGGGSGGGGSDIVLTQSPASLDVSLGQRATISCRASQSVSTSSYSYMHWYQQKPGQSPKLLIKYASNLESGVPARFSGSGSGTDFTLNIHPVEEEDTATYYCQHSWEIWTFGGGTKLEIKSGTTTPAPRPPTPAPTIASQPLSLRPEACRPAAGGAVHTRGLDFACDIFWVLVVVGGVLACYSLLVTVAFIIFWVRSKRSRLLHSDYMNMTPRRPGPTRKHYQPYAPPRDFAAYRSIDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRVNGSGATNFSLLKQAGDVEENPGPTSATMLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGER

SEQ ID NO: 15 - DNA, 69 bp, synthetic construct
  source 1..69: mol_type = other DNA
atggccctgcctgtgacagccctgctgctgcctctggctctgctgctgcatgccgccagacctggatct

SEQ ID NO: 16 - DNA, 60 bp, synthetic construct
  source 1..60: mol_type = other DNA
ggcggcggaggatctggcggaggcggaagtggcgggggaggaagcggagggggcggatct

SEQ ID NO: 17 - DNA, 138 bp, synthetic construct
  source 1..138: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatc

SEQ ID NO: 18 - DNA, 69 bp, synthetic construct
  source 1..69: mol_type = other DNA
tacatctgggcgcccttggccgggacttgtggggtccttctcctgtcactggttatcaccctttactgc

SEQ ID NO: 19 - DNA, 336 bp, synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 20 - DNA, 66 bp, synthetic construct
  source 1..66: mol_type = other DNA
ggaagcggcgccaccaacttcagcctgctgaaacaggccggcgacgtggaagagaaccctggcccc

SEQ ID NO: 21 - DNA, 975 bp, synthetic construct
  source 1..975: mol_type = other DNA
gccaccatgctcgtgcggagaggcgccagagctggccccagaatgcctagaggatggaccgccctgtgcctgctgagcctgctgcctagcggcttcatgagcctggacaacaacggcaccgccacccctgagctgcctacccagggcaccttcagcaacgtgtccaccaatgtgtcctaccaggaaaccaccacccccagcaccctgggcagcacatctctgcaccctgtgtcccagcacggcaacgaggccaccaccaacatcaccgagacaaccgtgaagttcaccagcaccagcgtgatcacctccgtgtacggcaacaccaacagcagcgtgcagagccagacctccgtgatcagcaccgtgtttaccacccccgctaatgtgtccacccccgaaaccaccctgaagcccagcctgtctcccggaaacgtgtccgacctgagcaccacctctaccagcctggccaccagccccaccaagccttacacaagcagcagccccatcctgagcgacatcaaggccgagatcaagtgcagcggcatccgggaagtgaagctgacacagggcatctgcctggaacagaacaagaccagcagctgcgccgagttcaagaaggacagaggcgagggcctggccagagtgctgtgtggcgaagaacaggccgatgccgatgctggcgctcaagtgtgctctctgctgctggcccagagcgaagtgcggcctcagtgcctgctgctggtgctggccaacagaaccgagatcagcagcaaactgcagctgatgaagaagcaccagagcgacctgaagaagctgggcatcctggacttcaccgagcaggacgtggcctcccaccagagctacagccagaaaaccctgatcgccctcgtgaccagcggagccctgctggcagtgctgggaatcaccggctactttctgatgaaccggcggagctggtcccccaccggcgaaaga

SEQ ID NO: 22 - protein, 23 aa, synthetic construct
MALPVTALLLPLALLLHAARPGS

SEQ ID NO: 23 - protein, 20 aa, synthetic construct
GGGGSGGGGSGGGGSGGGGS

SEQ ID NO: 24 - protein, 45 aa, synthetic construct
TTTPAPRPPTPAPTIASQPLSLRPEACRPAAGGAVHTRGLDFACD

SEQ ID NO: 25 - protein, 24 aa, synthetic construct
IYIWAPLAGTCGVLLLSLVITLYC

SEQ ID NO: 26 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 22 aa, synthetic construct
GSGATNFSLLKQAGDVEENPGP

SEQ ID NO: 28 - protein, 325 aa, synthetic construct
ATMLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGER

SEQ ID NO: 29 - DNA, 78 bp, synthetic construct
  source 1..78: mol_type = other DNA
tgggtgctggtggtggttggtggagtcctggcttgctatagcttgctagtaacagtggcctttattattttctgggtg

SEQ ID NO: 30 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 31 - protein, 27 aa, synthetic construct
FWVLVVVGGVLACYSLLVTVAFIIFWV

(19) United States
(12) Patent Application Publication
(10) Pub. No.: US 2023/0183315 A1
(43) Pub. Date: Jun. 15, 2023

(54) Regulatory T Cells Expressing Chimeric Antigen Receptors and Uses in Synucleinopathies

(71) Applicant: THE TRUSTEES OF THE UNIVERSITY OF PENNSYLVANIA, Philadelphia, PA (US)

(72) Inventors: Saar Gill, Philadelphia, PA (US); Nicholas Petty, Philadelphia, PA (US); Matias Porras Paniagua, Philadelphia, PA (US)

(21) Appl. No.: 17/938,258

(22) Filed: Oct. 5, 2022

Related U.S. Application Data
(60) Provisional application No. 63/253,007, filed on Oct. 6, 2021.

(51) Int. Cl. C07K 14/725; A61K 35/17; C07K 14/705

(52) U.S. Cl. CPC C07K 14/7051; A61K 35/17; C07K 14/70521; C07K 2319/03

(57) ABSTRACT

The present invention relates to compositions and methods for using regulatory T cells expressing chimeric antigen receptors for treating α-synucleinopathies such as Parkinson's disease. Also included are methods and pharmaceutical compositions comprising the modified regulatory T cells for treating Parkinson's disease.

### CROSS-REFERENCE TO RELATED APPLICATION

The present application is entitled to priority under 35 U.S.C. § 119(e) to U.S. Provisional Patent Application No. 63/253,007, filed Oct. 6, 2021, which is hereby incorporated by reference in its entirety herein.

### SEQUENCE LISTING

The present application contains a Sequence Listing which has been submitted in XML format via Patent Center and is hereby incorporated by reference in its entirety. Said XML file, created on Oct. 5, 2022, is named 046483_7311US1_SequenceListingST26.XML and is 37,824 bytes in size.

### BACKGROUND OF THE INVENTION

Parkinson's Disease (PD) is a progressive disorder of the central nervous system, characterized by resting tremors, shuffling gait, and cognitive decline, which affects 10 million people worldwide. Pathologically, PD is defined by the loss of dopaminergic (DA) neurons in the substantia nigra (SN) and the formation of intraneuronal alpha-synuclein fibrils into Lewy bodies. Neuroinflammation is increasingly recognized in PD and other neurodegenerative disorders. There are several palliative treatments for PD including dopamine replacement, Levodopa, or monoamine oxidase B inhibitors. However, these treatments are unable to halt disease progression or restore regulative cognitive or motor function.

Therefore a need exists for treatments for PD that can halt disease progression and/or restore regulative cognitive or motor function. The present invention addresses this need.

### SUMMARY OF THE INVENTION

As described herein, the present invention relates to compositions and methods for regulatory T cells expressing Chimeric Antigen Receptors (CARs) and uses in synucleinopathies.

In one aspect, the invention includes an isolated nucleic acid encoding a chimeric antigen receptor (CAR), the CAR comprising an antigen binding domain, a transmembrane domain, and an intracellular domain, wherein the antigen binding domain specifically binds to α-synuclein.

In another aspect, the invention includes a chimeric antigen receptor (CAR) comprising an antigen binding domain, a transmembrane domain, and an intracellular domain, wherein the antigen binding domain specifically binds to α-synuclein.

Another aspect of the invention includes a genetically modified cell comprising any of the CARs contemplated herein.

In various embodiments of the above aspects or any other aspect of the invention delineated herein, the antigen binding domain comprises a heavy chain variable (VH) domain encoded by a nucleic acid sequence at least 85%, 90%, 95%, 96%, 97%, 98%, 99%, or 100% identical to SEQ ID NO: 1, and/or a light chain variable domain (VL) domain encoded by a nucleic acid sequence at least 85%, 90%, 95%, 96%, 97%, 98%, 99%, or 100% identical to SEQ ID NO: 2.

In certain embodiments, the intracellular domain comprises a signaling domain and/or an intracellular domain of a costimulatory molecule. In certain embodiments, the signaling domain is a CD3 zeta signaling domain. In certain embodiments, the costimulatory molecule is CD28. In certain embodiments, the intracellular domain comprises a CD3 zeta signaling domain and a CD28 costimulatory molecule.

In certain embodiments, the CAR is encoded by a nucleic acid sequence at least 85%, 90%, 95%, 96%, 97%, 98%, 99%, or 100% identical to SEQ ID NO: 11 or SEQ ID NO: 13.

In certain embodiments, the antigen binding domain comprises a heavy chain variable (VH) domain comprising HCDR3, HCDR2, and HCDR1 regions, and a light chain variable (VL) domain comprising LCDR3, LCDR2 and LCDR1 regions. The HCDR3 region comprises the amino acid sequence AAEAY (SEQ ID NO: 5) and/or; the HCDR2 region comprises the amino acid sequence IDPENDNT (SEQ ID NO: 6) and/or; the HCDR1 region comprises the amino acid sequence GLNIKDYY (SEQ ID NO: 7) and/or; the LCDR3 region comprises the amino acid sequence QHSWEIWT (SEQ ID NO: 8) and/or; the LCDR2 region comprises the amino acid sequence YAS and/or; the LCDR1 region comprises the amino acid sequence QSVSTSSYSY (SEQ ID NO: 10).

In certain embodiments, the antigen binding domain comprises a heavy chain variable (VH) domain comprising an amino acid sequence least 85%, 90%, 95%, 96%, 97%, 98%, 99%, or 100% identical to SEQ ID NO: 3, and/or a light chain variable domain (VH) domain comprising an amino acid sequence least 85%, 90%, 95%, 96%, 97%, 98%, 99%, or 100% identical to SEQ ID NO: 4.

In certain embodiments, the CAR comprises an amino acid sequence at least 85%, 90%, 95%, 96%, 97%, 98%, 99%, or 100% identical to SEQ ID NO: 12 or SEQ ID NO: 14.

In certain embodiments, the cell is a regulatory T cell (Treg).

Another aspect of the invention includes a genetically modified regulatory T cell comprising a CAR, wherein the CAR comprises: i) an antigen binding domain comprising a heavy chain variable (VH) domain comprising HCDR3, HCDR2, and HCDR1 regions, and a light chain variable (VL) domain comprising LCDR3, LCDR2 and LCDR1 regions, wherein the HCDR3 region comprises the amino acid sequence AAEAY (SEQ ID NO: 5) and/or; the HCDR2 region comprises the amino acid sequence IDPENDNT (SEQ ID NO: 6) and/or; the HCDR1 region comprises the amino acid sequence GLNIKDYY (SEQ ID NO: 7) and/or; the LCDR3 region comprises the amino acid sequence QHSWEIWT (SEQ ID NO: 8) and/or; the LCDR2 region comprises the amino acid sequence YAS and/or; the LCDR1 region comprises the amino acid sequence QSVSTSSYSY (SEQ ID NO: 10); and ii) an intracellular domain comprising a CD3 zeta signaling domain and a CD28 costimulatory molecule.

Another aspect of the invention includes a pharmaceutical composition comprising any of the cells contemplated herein, and a pharmaceutically acceptable carrier.

Another aspect of the invention includes a method of generating a modified cell, the method comprising introducing into a cell any of the nucleic acids contemplated herein.

In certain embodiments, the cell is a regulatory T cell (Treg). In certain embodiments, the regulatory T cell is obtained by isolating CD4+CD25+ T cells from a population of cells. In certain embodiments, the regulatory T cell is obtained by modifying a T cell to express FOXP3.

Another aspect of the invention includes a method of promoting α-synuclein specific immunosuppression in a subject, the method comprising administering to the subject an effective amount of a regulatory T cell comprising a CAR, wherein the CAR comprises an antigen binding domain, a transmembrane domain, and an intracellular domain, wherein the antigen binding domain specifically binds to α-synuclein.

Another aspect of the invention includes a method of treating a neurodegenerative disease or a disease associated with an α-synucleinopathy in a subject, the method comprising administering to the subject an effective amount of a regulatory T cell comprising a CAR, wherein the CAR comprises an antigen binding domain, a transmembrane domain, and an intracellular domain, wherein the antigen binding domain specifically binds to α-synuclein.

In certain embodiments, the antigen binding domain comprises a heavy chain variable (VH) domain comprising HCDR3, HCDR2, and HCDR1 regions, and a light chain variable (VL) domain comprising LCDR3, LCDR2 and LCDR1 regions, wherein the HCDR3 region comprises the amino acid sequence AAEAY (SEQ ID NO: 5) and/or; the HCDR2 region comprises the amino acid sequence IDPENDNT (SEQ ID NO: 6) and/or; the HCDR1 region comprises the amino acid sequence GLNIKDYY (SEQ ID NO: 7) and/or; the LCDR3 region comprises the amino acid sequence QHSWEIWT (SEQ ID NO: 8) and/or; the LCDR2 region comprises the amino acid sequence YAS and/or; the LCDR1 region comprises the amino acid sequence QSVSTSSYSY (SEQ ID NO: 10).

In certain embodiments, the antigen binding domain comprises a heavy chain variable (VH) domain comprising an amino acid sequence least 85%, 90%, 95%, 96%, 97%, 98%, 99%, or 100% identical to SEQ ID NO: 3, and/or a light chain variable domain (VH) domain comprising an amino acid sequence least 85%, 90%, 95%, 96%, 97%, 98%, 99%, or 100% identical to SEQ ID NO: 4.

In certain embodiments, the antigen binding domain comprises a heavy chain variable (VH) domain encoded by a nucleic acid sequence at least 85%, 90%, 95%, 96%, 97%, 98%, 99%, or 100% identical to SEQ ID NO: 1, and/or a light chain variable domain (VH) domain encoded by a nucleic acid sequence at least 85%, 90%, 95%, 96%, 97%, 98%, 99%, or 100% identical to SEQ ID NO: 2.

In certain embodiments, the intracellular domain comprises a signaling domain and/or an intracellular domain of a costimulatory molecule. In certain embodiments, the signaling domain comprises a CD3 zeta signaling domain. In certain embodiments, the costimulatory molecule is CD28. In certain embodiments, the intracellular domain comprises a CD3 zeta signaling domain and a CD28 costimulatory molecule.

In certain embodiments, the CAR comprises an amino acid sequence at least 85%, 90%, 95%, 96%, 97%, 98%, 99%, or 100% identical to SEQ ID NO: 12 or SEQ ID NO: 14.

In certain embodiments, the CAR is encoded by a nucleic acid sequence at least 85%, 90%, 95%, 96%, 97%, 98%, 99%, or 100% identical to SEQ ID NO: 11 or SEQ ID NO: 13.

In certain embodiments, the subject is human.

In certain embodiments, the disease is selected from Parkinson's disease (PD), dementia with Lewy bodies (DLB), multiple system atrophy (MSA), and Alzheimer's disease with mixed Lewy pathology.

In certain embodiments, the disease is Parkinson's disease (PD).

### BRIEF DESCRIPTION OF THE DRAWINGS

The following detailed description of preferred embodiments of the invention will be better understood when read in conjunction with the appended drawings. For the purpose of illustrating the invention, there are shown in the drawings certain embodiments of the invention. It should be understood, however, that the invention is not limited to the precise arrangements and instrumentalities of the embodiments shown in the drawings.

FIGS. 1A-1C depict schematic diagrams showing anti-α-synuclein CAR constructs and nucleic acid and protein sequences of the constructs. FIG. 1A: the α-synuclein CAR constructs generated: first generation α synuclein targeting CAR (top); and second generation a synuclein targeting CAR comprising the co-stimulatory CD28 domain (bottom). FIG. 1B: first generation α synuclein targeting CAR, indicating the position of the VH and VL regions of the α-synuclein binding domain. FIG. 1C: second generation α-synuclein targeting CAR construct, indicating the positions of the VH and VL regions of the α-synuclein binding domain.

FIG. 2 is a diagram showing a process for making the nASCAR Tregs.

FIGS. 3A-3C depict FACS scan analyses and quantitation of the data showing confirmation of transduction by staining for CD34 (here used as a reporter tag) and confirmation of the Treg phenotype by staining for CD4, FOXP3 and CD25, along with a comparison with CD4 T conventional (Tconv) cells. FIG. 3A: FACS scan of CD4 vs CD34. FIG. 3B: FACS scan of CD25 vs FOXP3. FIG. 3C: Quantitation of the percent CD4+CD25+FOXP3+ cells in AS CAR generation 1 (Gen1), in AS CAR generation 2 (Gen2), and in CD19 CAR (CAR19).

FIGS. 4A-4D show surface expression of LAP (FIG. 4A), CD39 (FIG. 4B), CD69 (FIG. 4C), and CTLA4 (FIG. 4D) on nASCAR Tregs and on control cells at rest or upon non-specific stimulation or specific stimulation with AS antigen.

FIGS. 5A-5F show levels of TNF-α (FIG. 5A), IL-10 (FIG. 5B), IL-6 (FIG. 5C), IL-4 (FIG. 5D), IFN-γ (FIG. 5E) and IL-2 (FIG. 5F) produced by nASCAR Tregs and control cells at rest or upon non-specific stimulation or specific stimulation with AS antigen.

FIG. 6 depicts results of a suppression assay, showing that nASCAR Tconv proliferation upon exposure to α-synuclein PFF is inhibited in the presence of antigen-specific nASCAR Tregs but not in the presence of control anti-CD19 CAR Tregs. Proliferation assay (% suppression) representative donor (ND518) *** p<0.0005 **** p<0.00005.

FIGS. 7A-7F are a set of plots showing the effect of the antigen-specific nASCAR Tregs on the production of TNF-α (FIG. 7A), IL-2 (FIG. 7B), IL-4 (FIG. 7C), IL-6 (FIG. 7D), IL-10 (FIG. 7E) and IFN-γ (FIG. 7F) after 24 hours.

FIGS. 8A-8F are a set of plots showing the effect of the antigen-specific nASCAR Tregs on the production of TNF-α (FIG. 8A), IL-2 (FIG. 8B), IL-4 (FIG. 8C), IL-6 (FIG. 8D), IL-10 (FIG. 8E) and IFN-γ (FIG. 8F) after 120 hours. Antigen-specific nASCAR Tregs were superior to non-specific CAR Tregs in blocking the production of TNF, IL-10, IL-4 and IFN-γ.

FIGS. 9A-9B depict FACS scan analyses showing enrichment of human CD4+ T cells (FIG. 9A), followed by flow sorting of the CD4+CD25++ population (FIG. 9B).

FIG. 10 is a set of plots showing results of a suppression assay, showing that nASCAR Tconv proliferated extensively upon exposure to α-synuclein PFF, and that this was inhibited in the presence of antigen-specific nASCAR Tregs (top panels) but not in the presence of control anti-CD19 CAR Tregs (bottom panels).

FIGS. 11A-11E is a set of plots showing the release of TNF-α (FIG. 11A), IL-10 (FIG. 11B), IL-8 (FIG. 11C), IL-6 (FIG. 11D), IL-1B (FIG. 11E) by primary human macrophages from three different donors upon exposure to α-synuclein PFF or control conditions.

FIG. 12 is a schematic showing injection of immunodeficient NSG mice with α-synuclein PFF followed by nASCAR or control T cells given intravenously.

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. α and β light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “auto-antigen” means, in accordance with the present invention, any self-antigen which is recognized by the immune system. Auto-antigens comprise, but are not limited to, cellular proteins, phosphoproteins, cellular surface proteins, cellular lipids, nucleic acids, glycoproteins, including cell surface receptors.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “chimeric antigen receptor” or “CAR,” as used herein, refers to an artificial T cell receptor that is engineered to be expressed on an immune effector cell and specifically bind an antigen. CARs may be used as a therapy with adoptive cell transfer. T cells are removed from a patient and modified so that they express the receptors specific to a particular form of antigen. In some embodiments, the CARs have been expressed with specificity for a tumor associated antigen, for example. CARs may also comprise an intracellular activation domain, a transmembrane domain and an extracellular domain comprising a tumor associated antigen binding region. In some aspects, CARs comprise fusions of single-chain variable fragments (scFv) derived monoclonal antibodies, fused to CD3-zeta transmembrane and intracellular domain. The specificity of CAR designs may be derived from ligands of receptors (e.g., peptides). In some embodiments, a CAR can target a disease by redirecting the specificity of a T cell expressing the CAR specific for a disease associated antigens.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

“Co-stimulatory ligand,” as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA a Toll ligand receptor, and any molecule that binds to those listed elsewhere herein.

A “co-stimulatory signal”, as used herein, refers to a signal, which in combination with a primary signal, such as TCR/CD3 ligation, leads to T cell proliferation and/or upregulation or downregulation of key molecules.

The term “derived from” refers to being generated, synthesized, or originating from a particular source, such that the derived matter is related to the source. The derived matter does not need to be identical to the particular source. In one embodiment, an antigen is derived from a protein. In another embodiment, a single-chain variable fragment is derived from a monoclonal antibody.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “neurodegenerative disease” as used herein, refers to a neurological disease characterized by loss or degeneration of neurons and by the presence of misfolded protein aggregates in the cytoplasm and/or nucleus of nerve cells or in the extracellular space (Forman et al., Nat. Med. 10, 1055 (2004)). Neurodegenerative diseases include neurodegenerative movement disorders and neurodegenerative conditions relating to memory loss and/or dementia. Neurodegenerative diseases include tauopathies and α-synucleopathies. Examples of neurodegenerative diseases include, but are not limited to presenile dementia, senile dementia, Alzheimer's disease, Parkinsonism linked to chromosome 17 (FTDP-17), progressive supranuclear palsy (PSP), Pick's disease, primary progressive aphasia, frontotemporal dementia, corticobasal dementia, Parkinson's disease, Parkinson's disease with dementia, dementia with Lewy bodies, Down's syndrome, multiple system atrophy, amyotrophic lateral sclerosis (ALS), Hallervorden-Spatz syndrome, polyglutamine disease, trinucleotide repeat disease, and prion disease.

“Effective amount” or “therapeutically effective amount” are used interchangeably herein, and refer to an amount of a compound, formulation, material, or composition, as described herein effective to achieve a particular biological result or provides a therapeutic or prophylactic benefit.

“Encoding” refers to the inherent property of specific sequences of nucleotides in a polynucleotide, such as a gene, a cDNA, or a RNA like mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

The phrases “an immunologically effective amount”, “an anti-immune response effective amount”, “an immune response-inhibiting effective amount”, or “therapeutic amount” refer to the amount of the composition of the present invention to be administered to a subject which amount is determined by a physician, optionally in consultation with a scientist, in consideration of individual differences in age, weight, immune response, type of disease/condition, and the health of the subject (patient) so that the desired result is obtained in the subject.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The terms “Lewy body”, “Lewy bodies”, and “Lewy neurites”, refer to abnormal aggregates of protein that develop in nerve cells. The primary protein aggregate in a Lewy body or Lewy neurite is composed of α-synuclein.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “protein aggregate” as used herein means two or more proteins that have aggregated together in a tissue in a subject to give rise to a pathological condition, or which places the subject at risk for a pathological condition. Non-limiting examples of such protein aggregates include aggregates of amyloid protein, aggregates of α-synuclein protein, aggregates of tau protein, aggregates of TDP-43 protein, aggregates of immunoglobulin light chains or transthyretin protein, aggregates of prion protein and the like.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell. An example of a “cell surface receptor” is human FSHR.

“Similarity” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are similar at that position. The similarity between two sequences is a direct function of the number of matching or similar positions; e.g., if half (e.g., five positions in a polymer ten subunits in length) of the positions in two sequences are similar, the two sequences are 50% similar; if 90% of the positions (e.g., 9 of 10), are matched or similar, the two sequences are 90% similar.

“Single chain antibodies” refer to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to compositions and methods for using T cells, in particular, regulatory T cells (“Tregs”) expressing chimeric antigen receptors, for treating diseases or conditions associated with the aggregation of α-synuclein, such as Parkinson's Disease (“PD”).

Observations from patients with PD that implicate the immune system in disease pathogenesis exist, including a provocative report on alpha synuclein (“aSyn”)-specific T cells in patients with PD (Sulzer et al., Nature, 2017 Jun. 28; 546(7660):656-661). These are supported by observations from mouse models of the disease. Direct evidence that aSyn can induce PD pathology and that its blockade can prevent pathology (Luk et al., Science, 2012 Nov. 16; 338(6109):949-53) have led to interest in the development of therapeutics targeting aSyn. Herein, it was hypothesized that aSyn specific immunosuppression could halt the progression of PD by inhibiting the spread of aSyn while dampening pathologic glial activation.

Described herein is a study which brought together advances in adoptive cell therapy, regulatory T cell (Treg) engineering, and neuroimmunology by creating anti-aSyn Tregs. In addition to the role that glia play in PD-associated neuroinflammation, T cells from the peripheral immune system have been implicated as well. Not only have T cells been observed in the brains of PD patients, but AS-reactive T cells have been found in their peripheral blood. Both MHC class I and class II have been found to play a role in mediating PD disease progression, potentially acting in conjunction with these immunoreactive cells in the brain. On the other end of the spectrum, regulatory T-cells have been shown to have a neuroprotective effect in murine models of PD. All of these findings indicate that both resident and systemic immune cells play a major role in the progression of PD, presenting an attractive new therapeutic target.

Cellular immunotherapies are an emerging new field of treatments, particularly in the field of oncology. Immune cells, such as T cells and macrophages, have been redirected to detect and eliminate cancer cells using chimeric antigen receptors (CARs). In 2017, CAR T cells targeted against CD19 (Kymriah & Yescarta) received FDA approval as an alternative to chemotherapy. More recently, several groups have shown that cells engineered with CARs can be employed in other contexts such as CAR T cells directed against fibroblasts to treat cardiac fibrosis, autoreactive B cells to treat pemphigus vulgaris, and regulatory CAR T cells to help prevent graft rejection. Given the recent discoveries implicating neuroinflammation in PD progression, it was reasoned that regulatory T cells (Tregs) could be engineered to hone to regions of increased pathology using an AS-directed CAR (nASCAR) and reduce DA neuron death, slowing or halting the progression of PD.

Human T cells can be engineered into CAR Tregs by genetic co-expression of the canonical Treg transcription factor FOXP3 with the chimeric antigen receptor of interest, or by sorting CD4+CD25++ T cells followed by transduction with the CAR. The latter approach was selected for further study. Regulatory or conventional T cells directed against aSyn were made by cloning the sequence of anti-aSyn monoclonal antibody into a standard lentiviral expression vector, and were compared with anti-CD19 CAR T cell controls. Expansion kinetics, CAR expression and surface phenotype showed that CAR expression was reproducibly higher on Tregs than Tconv. Upon stimulation with aSyn peptides, anti-aSyn CAR Tregs produced immunosuppressive cytokines (IL-10) whereas anti-aSyn CAR T conv produced inflammatory cytokines (IL2, IFNγ).

Exposure to aSyn peptides led to extensive proliferation and production of cytokines by anti-aSyn CARTconv cells, and this was almost completely blocked in the presence of anti-aSyn CARTregs. These results provide a proof-of-concept for the application of a CAR Treg based cellular immunotherapy for the treatment of PD.

### Alpha-Synuclein

Parkinson's disease is a neurodegenerative disorder that is pathologically characterized by the presence of intracytoplasmic Lewy bodies and Lewy neurites (Lewy in Handbuch der Neurologie, M. Lewandowski, ed., Springer, Berlin, pp. 920-933, 1912; Pollanen et al., J. Neur opath. Exp. Neurol. 52:183-191, 1993), the major components of which are filaments consisting of α-synuclein (Spillantini et al., Proc. Natl. Acad. Sci. USA 95:6469-6473, 1998; Arai et al., Neurosc. Lett. 259:83-86, 1999), a 140-amino acid protein (Ueda et al., Proc. Natl. Acad. Sci. U.S.A. 90:11282-11286, 1993). Accumulation of α-synuclein is also a cytopathological feature common to Lewy body disease and multiple system atrophy (Wakabayashi et al, Acta Neuropath. 96:445-452, 1998; Piao et al, Acta Neuro path. 101:285-293, 2001). Multiple system atrophy is a sporadic neurodegenerative disease in adults characterized by neuronal and glial cytoplasmic inclusions, containing α-synuclein. Parkinson's disease α-synuclein fibrils, like the Aβ amyloid fibrils of Alzheimer's disease, also consist of a predominant beta-pleated sheet structure. α-Synucleinopathies are conditions associated with the aggregation of α-synuclein and include Parkinson's disease, LB variant Alzheimer's disease, multiple system atrophy (MSA), LB dementia and Hallervorden-Spatz disease.

The compositions and methods disclosed herein use an antibody against an alpha-synuclein polypeptide. The terms “alpha synuclein”, “α-syn,” “aSyn,” and “alpha-syn” are used interchangeably herein. α-syn encompasses naturally occurring alpha synuclein sequences (e.g., naturally occurring human wild type and mutant alpha synucleins) as well as functional variants thereof. Unless otherwise apparent from the context, reference to α-Syn or its fragments includes the natural human amino acid sequence indicated elsewhere herein, or fragments thereof, as well as analogs including allelic, species and induced variants. Amino acids of analogs are assigned the same numbers as corresponding amino acids in the natural human sequence when the analog and human sequence are maximally aligned. Analogs typically differ from naturally occurring peptides at one, two or a few positions, often by virtue of conservative substitutions. Some natural allelic variants are genetically associated with hereditary PD and LBD. The term “allelic variant” is used to refer to variations between genes of different individuals in the same species and corresponding variations in proteins encoded by the genes. α-Syn, its fragments, and analogs can be synthesized by solid phase peptide synthesis or recombinant expression, or can be obtained from natural sources. Automatic peptide synthesizers are commercially available from numerous suppliers, such as Applied Biosystems, Foster City, Calif.

### Chimeric Antigen Receptor (CAR) Composition

In one aspect, the invention includes an isolated nucleic acid sequence encoding a chimeric antigen receptor (CAR). The CAR of the invention comprises an antigen binding domain, a transmembrane domain and an intracellular domain, wherein the antigen binding domain specifically binds to α-synuclein. In one embodiment, intracellular domain comprises a signaling domain and/or an intracellular domain of a co-stimulatory molecule.

In another aspect, the invention includes a CAR comprising an antigen binding domain, a transmembrane domain and an intracellular domain of a co-stimulatory molecule and a signaling domain, wherein the antigen binding domain comprises an antibody that is capable of binding a protein in a protein aggregate, in a tissue of a subject with a disease associated with α-synuclein aggregation or synucleinopathies.

In yet another aspect, the invention includes a vector comprising the CAR as described elsewhere herein.

In one embodiment, the antigen binding domain comprises a heavy and light chain. In one embodiment, the antigen binding domain is an antibody, e.g., an alpha-synuclein antibody. In another embodiment, the alpha-synuclein antibody is Syn303. In yet another embodiment, the antigen binding domain is selected from the group consisting of a Fab fragment, a F(ab′)₂ fragment, a Fv fragment, and a single chain Fv (scFv).

In one embodiment, the disease is selected from Parkinson's disease (PD), dementia with Lewy bodies (DLB, also known as Lewy body dementia), multiple system atrophy (MSA), and Alzheimer's disease with mixed Lewy pathology.

In one embodiment, the disease is associated with an α-synucleinopathy. In one embodiment, the disease is a neurodegenerative disease. In one embodiment, the disease is Parkinson's disease (PD).

In one embodiment, the costimulatory signaling region comprises an intracellular domain of a costimulatory molecule selected from the group consisting of CD8, CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof. In another embodiment, the signaling domain is CD3zeta.

In one embodiment, the nucleic acid sequence is selected from the group consisting of a DNA and an mRNA. In another embodiment, the nucleic acid sequence encoding the CAR is codon optimized to favor an increase in gene expression, translation efficiency and/or protein expression.

In one aspect of the invention, a modified T cell is generated by expressing the CAR described elsewhere herein. Thus, the present invention encompasses a CAR and a nucleic acid construct encoding a CAR, wherein the CAR includes an antigen binding domain, a transmembrane domain and an intracellular domain.

In one aspect, the invention includes a modified cell comprising a CAR, wherein the CAR comprises an antigen binding domain, a transmembrane domain and an intracellular domain of a co-stimulatory molecule and wherein the antigen binding domain comprises an antibody that is capable of binding alpha-synuclein in a tissue of a subject with Parkinson's disease. In another aspect, the invention includes a modified cell comprising a nucleic acid sequence encoding a CAR, wherein nucleic acid sequence comprises a nucleic acid sequence encoding an antigen binding domain, a nucleic acid sequence encoding a transmembrane domain and a nucleic acid sequence encoding an intracellular domain of a co-stimulatory molecule, wherein the antigen binding domain comprises an antibody that is capable of binding alpha-synuclein in a tissue of a subject with Parkinson's disease and wherein the cell is a regulatory T cell (Treg).

Chimeric Antigen Receptor

The present invention also includes a composition comprising a CAR. The CAR comprises an antigen binding domain, a transmembrane domain and an intracellular domain of a co-stimulatory molecule.

Example of CARs are described in U.S. Pat. Nos. 8,911,993, 8,906,682, 8,975,071, 8,916,381, 9,102,760, 9,101,584, and 9,102,761, all of which are incorporated herein by reference in their entireties.

In some embodiments, the CAR comprises an antigen binding domain that binds to an antigen on a target cell. Examples of cell surface markers that may act as an antigen that binds to the antigen binding domain of the CAR include those associated with viral, bacterial and parasitic infections, autoimmune disease, and cancer cells.

The choice of antigen binding domain depends upon the type and number of antigens that are present on the surface of a target cell. For example, the antigen binding domain may be chosen to recognize an antigen that acts as a cell surface marker on a target cell associated with a particular disease state. In one embodiment, the antigen is α-synuclein.

In other embodiments, the antigen binding domain of the CAR is capable of binding to an antigen and the variable heavy chain fragment and the variable light chain fragment of the minibody bind the same antigen.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. Thus, in some embodiments, the antigen binding domain portion of the CAR comprises a mammalian antibody or a fragment thereof.

In some embodiments, the antigen binding domain of the α-synuclein CAR comprises a heavy chain variable (VH) domain and a light chain variable (VL) domain.

In some embodiments, the heavy chain variable (VH) domain of the antigen binding domain of the α-synuclein CAR is encoded by a nucleic acid sequence comprising the sequence set forth in SEQ ID NO: 1:

(GAGGTGCAGCTGCAGCAGTCTGGCGCTGAACTCGTGCGGCCTGGCGCTC
TCGTGAAGCTGAGCTGTAAAGCCAGCGGCCTGAACATCAAGGACTACTAC
ATGCACTGGGTCAAGCAGCGGCCCGAGCAGGGCCTGGAATGGATCGGCTG
GATCGACCCCGAGAACGACAACACCAGATTCGACCCCCGGTTCCAGGGCC
GGGTGTCCATCATTGCCGACACCAGCAGCAACACCGCCTACCTGCAGCTG
TCCAGCCTGACCAGCGAGGACACCGCCGTGTACTATTGTGCCGCCGAGGC
CTATTGGGGCCAGGGCACACTCGTGACAGTGTCTGCT).

In some embodiments, the light chain variable (VL) domain of the antigen binding domain of the α-synuclein CAR is encoded by a nucleic acid sequence comprising the sequence set forth in SEQ ID NO: 2:

(GATATCGTGCTGACACAGAGCCCCGCCTCCCTGGATGTGTCCCTGGGAC
AGAGAGCCACAATCAGCTGCAGAGCCTCCCAGAGCGTGTCCACCAGCTCC
TACAGCTATATGCACTGGTATCAGCAGAAGCCCGGCCAGAGCCCCAAGCT
GCTGATTAAGTACGCCAGCAACCTGGAAAGCGGCGTGCCCGCCAGATTTT
CTGGCAGCGGCTCTGGCACCGACTTCACCCTGAATATCCACCCCGTGGAA
GAGGAAGATACCGCCACCTACTACTGCCAGCACAGCTGGGAGATCTGGAC
CTTCGGCGGAGGCACCAAGCTGGAAATCAAG)

In some embodiments, the heavy chain variable (VH) domain of the antigen binding domain of the α-synuclein CAR comprises the amino acid sequence set forth in SEQ ID NO: 3:

(EVQLQQSGAELVRPGALVKLSCKASGLNIKDYYMHWVKQRPEQGLEWIG
WIDPENDNTRFDPRFQGRVSIIADTSSNTAYLQLSSLTSEDTAVYYCAAE
AYWGQGTLVTVSA).

In some embodiments, the light chain variable (VL) domain of the antigen binding domain of the α-synuclein CAR comprises the amino acid sequence set forth in SEQ ID NO: 4:

(DIVLTQSPASLDVSLGQRATISCRASQSVSTSSYSYMHWYQQKPGQSPK
LLIKYASNLESGVPARFSGSGSGTDFTLNIHPVEEEDTATYYCQHSWEIW
TFGGGTKLEIK).

In some embodiments, the α-synuclein CAR comprises an antigen binding domain comprising a heavy chain variable (VH) domain comprising HCDR3, HCDR2, and HCDR1 regions, and a light chain variable (VL) domain comprising LCDR3, LCDR2 and LCDR1 regions.

In some embodiments, the HCDR3 region comprises the amino acid sequence set forth in SEQ ID NO: 5 (AAEAY). In some embodiments, the HCDR2 region comprises the amino acid sequence set forth in SEQ ID NO: 6 (IDPENDNT). In some embodiments, the HCDR1 region comprises the amino acid sequence set forth in SEQ ID NO: 7 (GLNIKDYY).

In some embodiments, the LCDR3 region comprises the amino acid sequence set forth in SEQ ID NO: 8 (QHSWEIWT). In some embodiments, the LCDR2 region comprises the amino acid sequence YAS. In some embodiments, the LCDR1 region comprises the amino acid sequence set forth in SEQ ID NO: 10 (QSVSTSSYSY).

In some embodiments, the antigen binding domain specifically binds to α-synuclein. In some instances, the antigen binding domain is derived from the same species in which the CAR will ultimately be used. For example, for use in humans, it may be beneficial for the antigen binding domain of the CAR to comprise a human antibody, humanized antibody as described elsewhere herein, or a fragment thereof.

The antigen binding domain may be operably linked to another domain of the CAR, such as the transmembrane domain and/or the intracellular domain, both described elsewhere herein, for expression in the cell. In one embodiment, a nucleic acid encoding the antigen binding domain is operably linked to a nucleic acid encoding a transmembrane domain and a nucleic acid encoding an intracellular domain.

With respect to the transmembrane domain, the CAR can be designed to comprise a transmembrane domain that connects the antigen binding domain of the CAR to the intracellular domain. In one embodiment, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some instances, a variety of human hinges can be employed as well including the human Ig (immunoglobulin) hinge.

In one embodiment, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

The intracellular domain or otherwise the cytoplasmic domain of the CAR is responsible for activation of the cell in which the CAR is expressed. The term “intracellular domain” is thus meant to include any portion of the intracellular domain sufficient to transduce the activation signal. In one embodiment, the intracellular domain includes a domain responsible for an effector function. The term “effector function” refers to a specialized function of a cell. Effector function of a T cell, for example, may be cytolytic activity or helper activity including the secretion of cytokines.

In one embodiment, the intracellular domain of the CAR includes a domain responsible for signal activation and/or transduction. The intracellular domain may transmit signal activation via protein-protein interactions, biochemical changes or other response to alter the cell's metabolism, shape, gene expression, or other cellular response to activation of the chimeric intracellular signaling molecule.

Examples of an intracellular domain for use in the invention include, but are not limited to, the cytoplasmic portion of the T cell receptor (TCR) and any co-stimulatory molecule that acts in concert to initiate signal transduction following antigen receptor engagement, as well as any derivative or variant of these elements and any synthetic sequence that has the same functional capability. In one embodiment, the intracellular domain of the CAR comprises dual signaling domains. The dual signaling domains may include a fragment or domain from any of the molecules described herein.

Examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD162), LTBR, LAT, GADS, SLP-76, PAG/Cbp, NKp44, NKp30, NKp46, NKG2D, other co-stimulatory molecules described herein, any derivative, variant, or fragment thereof, any synthetic sequence of a co-stimulatory molecule that has the same functional capability, and any combination thereof.

In one embodiment, the intracellular domain of the CAR includes any portion of a co-stimulatory molecule, such as at least one signaling domain from CD3, CD27, CD28, ICOS, 4-1BB, PD-1, T cell receptor (TCR), any derivative or variant thereof, any synthetic sequence thereof that has the same functional capability, and any combination thereof.

Between the antigen binding domain and the transmembrane domain of the CAR, or between the intracellular domain and the transmembrane domain of the CAR, a spacer domain may be incorporated. As used herein, the term “spacer domain” generally means any oligo- or polypeptide that functions to link the transmembrane domain to, either the antigen binding domain or, the intracellular domain in the polypeptide chain. In one embodiment, the spacer domain may comprise up to 300 amino acids, for example, 10 to 100 amino acids, or 25 to 50 amino acids. In another embodiment, a short oligo- or polypeptide linker, from 2 and 10 amino acids in length may form the linkage between the transmembrane domain and the intracellular domain of the CAR. An example of a linker includes a glycine-serine doublet.

In some embodiments the nucleic acid sequence of the first generation α-synuclein CAR comprises a sequence provided by SEQ ID NO: 11. In some embodiments the amino acid sequence of the first generation α-synuclein CAR comprises a sequence provided by SEQ ID NO: 12. In some embodiments the nucleic acid sequence of the second generation α-synuclein CAR comprises a sequence provided by SEQ ID NO: 13. In some embodiments the amino acid sequence of the second generation α-synuclein CAR comprises a sequence provided by SEQ ID NO: 14.

Tolerable variations of the α-synuclein CAR construct or a functional portion thereof will be known to those of skill in the art. For example, in some embodiments the CAR construct comprises a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the nucleic sequences set forth in SEQ ID NOs: 1, 2, 11, or 13.

In some embodiments the α-synuclein CAR construct or a functional portion thereof is encoded by an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least or 99% sequence identity to the amino acid sequence set forth in SEQ ID NOs: 3, 4, 5, 6, 7, 8, 9, 10, 12, or 14.

Exemplary Nucleotide and Amino Acid Sequences:

First generation α-synuclein targeting CAR construct (SEQ ID NO: 11):
Domains are labeled as follows:
(SEQ ID NO: 15)
CD8 Leader
(SEQ ID NO: 1)
aSyn (VH)
(SEQ ID NO: 16)
ScFv linker
(SEQ ID NO: 2)
aSyn (VL)
(SEQ ID NO: 17)
CD8 Hinge
(SEQ ID NO: 18)

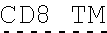

(SEQ ID NO: 19)
CD3z
(SEQ ID NO: 20)

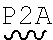

(SEQ ID NO: 21)
CD34 Truncated
ATGGCCCTGCCTGTGACAGCCCTGCTGCTGCCTCTGGCTCTGCTGCTGCATGCCGCCA
GACCTGGATCTGAGGTGCAGCTGCAGCAGTCTGGCGCTGAACTCGTGCGGCCTG
GCGCTCTCGTGAAGCTGAGCTGTAAAGCCAGCGGCCTGAACATCAAGGACTACT
ACATGCACTGGGTCAAGCAGCGGCCCGAGCAGGGCCTGGAATGGATCGGCTGG
ATCGACCCCGAGAACGACAACACCAGATTCGACCCCCGGTTCCAGGGCCGGGT
GTCCATCATTGCCGACACCAGCAGCAACACCGCCTACCTGCAGCTGTCCAGCCT
GACCAGCGAGGACACCGCCGTGTACTATTGTGCCGCCGAGGCCTATTGGGGCC
AGGGCACACTCGTGACAGTGTCTGCTGGCGGCGGAGGATCTGGCGGAGGCGGAAG
TGGCGGGGGAGGAAGCGGAGGGGGCGGATCTGATATCGTGCTGACACAGAGCCCC
GCCTCCCTGGATGTGTCCCTGGGACAGAGAGCCACAATCAGCTGCAGAGCCTC
CCAGAGCGTGTCCACCAGCTCCTACAGCTATATGCACTGGTATCAGCAGAAGCC
CGGCCAGAGCCCCAAGCTGCTGATTAAGTACGCCAGCAACCTGGAAAGCGGCG
TGCCCGCCAGATTTTCTGGCAGCGGCTCTGGCACCGACTTCACCCTGAATATCC
ACCCCGTGGAAGAGGAAGATACCGCCACCTACTACTGCCAGCACAGCTGGGAG
ATCTGGACCTTCGGCGGAGGCACCAAGCTGGAAATCAAGtccggaaccacgacgccagcgcc
gcgaccaccaacaccggcgcccaccatcgcgtcgcagcccctgtccctgcgcccagaggcgtgccggccagcggcggggggcgc
agtgcacacgagggggctggacttcgcctgtgatatcatc 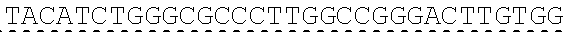 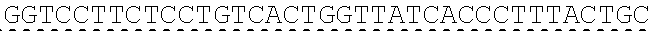
AGAGTGAAGTTCAGCAGGAGC
GCAGACGCCCCCGCGTACAAGCAGGGCCAGAACCAGCTCTATAACGAGCTCAATCTA
GGACGAAGAGAGGAGTACGATGTTTTGGACAAGAGACGTGGCCGGGACCCTGAGAT
GGGGGGAAAGCCGAGAAGGAAGAACCCTCAGGAAGGCCTGTACAATGAACTGCAG
AAAGATAAGATGGCGGAGGCCTACAGTGAGATTGGGATGAAAGGCGAGCGCCGGAG
GGGCAAGGGGCACGATGGCCTTTACCAGGGTCTCAGTACAGCCACCAAGGACACCTA
CGACGCCCTTCACATGCAGGCCCTGCCCCCTCGCGttaac 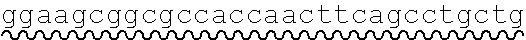 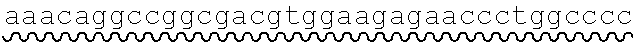
actagtgccaccatgctcgtgcggagaggcgccagagctggccccagaatg
cctagaggatggaccgccctgtgcctgctgagcctgctgcctagcggcttcatgagcctggacaacaacggcaccgccacccctgagctg
cctacccagggcaccttcagcaacgtgtccaccaatgtgtcctaccaggaaaccaccacccccagcaccctgggcagcacatctctgcac
cctgtgtcccagcacggcaacgaggccaccaccaacatcaccgagacaaccgtgaagttcaccagcaccagcgtgatcacctccgtgta
cggcaacaccaacagcagcgtgcagagccagacctccgtgatcagcaccgtgtttaccacccccgctaatgtgtccacccccgaaaccac
cctgaagcccagcctgtctcccggaaacgtgtccgacctgagcaccacctctaccagcctggccaccagccccaccaagccttacacaag
cagcagccccatcctgagcgacatcaaggccgagatcaagtgcagcggcatccgggaagtgaagctgacacagggcatctgcctggaa
cagaacaagaccagcagctgcgccgagttcaagaaggacagaggcgagggcctggccagagtgctgtgtggcgaagaacaggccgat
gccgatgctggcgctcaagtgtgctctctgctgctggcccagagcgaagtgcggcctcagtgcctgctgctggtgctggccaacagaacc
gagatcagcagcaaactgcagctgatgaagaagcaccagagcgacctgaagaagctgggcatcctggacttcaccgagcaggacgtgg
cctcccaccagagctacagccagaaaaccctgatcgccctcgtgaccagcggagccctgctggcagtgctgggaatcaccggctactttct
gatgaaccggcggagctggtcccccaccggcgaaaga
First generation α-synuclein targeting CAR construct (SEQ ID NO: 12):
Domains are labeled as follows:
(SEQ ID NO: 22)
CD8 Leader
(SEQ ID NO: 3)
aSyn (VH)
(SEQ ID NO: 23)
ScFv linker
(SEQ ID NO: 4)
aSyn (VL)
(SEQ ID NO: 24)
CD8 Hinge
(SEQ ID NO: 25)

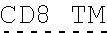

(SEQ ID NO: 26)
CD3z
(SEQ ID NO: 27)

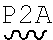

(SEQ ID NO: 28)
CD34 Truncated
MALPVTALLLPLALLLHAARPGSEVQLQQSGAELVRPGALVKLSCKASGLNIKDYYM
HWVKQRPEQGLEWIGWIDPENDNTRFDPRFQGRVSIIADTSSNTAYLQLSSLTSEDT
AVYYCAAEAYWGQGTLVTVSAGGGGSGGGGSGGGGSGGGGSDIVLTQSPASLDVSLG
QRATISCRASQSVSTSSYSYMHWYQQKPGQSPKLLIKYASNLESGVPARFSGSGSGT
DFTLNIHPVEEEDTATYYCQHSWEIWTFGGGTKLEIKSGTTTPAPRPPTPAPTIASQPLS
LRPEACRPAAGGAVHTRGLDFACDI 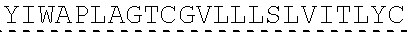 RVKFSRSADAPAYK
QGQNQLYNELNLGRREEYDVLDKRRGRDPEMGGKPRRKNPQEGLYNELQKDKMAEAY
SEIGMKGERRRGKGHDGLYQGLSTATKDTYDALHMQALPPRVN 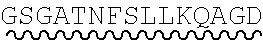 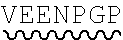
TSATMLVRRGARAGPRMPRGWTALCLLSLLPSGFMSLDNNGTATPELPTQGT
FSNVSTNVSYQETTTPSTLGSTSLHPVSQHGNEATTNITETTVKFTSTSVITSVYGNTNSS
VQSQTSVISTVFTTPANVSTPETTLKPSLSPGNVSDLSTTSTSLATSPTKPYTSSSPILSDIKA
EIKCSGIREVKLTQGICLEQNKTSSCAEFKKDRGEGLARVLCGEEQADADAGAQVCSLLL
AQSEVRPQCLLLVLANRTEISSKLQLMKKHQSDLKKLGILDFTEQDVASHQSYSQKTLIA
LVTSGALLAVLGITGYFLMNRRSWSPTGER
Second generation α-synuclein targeting CAR construct (SEQ ID NO: 13):
Domains are labeled as follows:
(SEQ ID NO: 22)
CD8 Leader
(SEQ ID NO: 1)
aSyn (VH)
(SEQ ID NO: 16)
ScFv linker
(SEQ ID NO: 2)
aSyn (VL)
(SEQ ID NO: 17)
CD8 Hinge
(SEQ ID NO: 29)

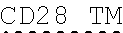

(SEQ ID NO: 30)

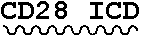

(SEQ ID NO: 19)
CD3z
(SEQ ID NO: 20)

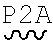

(SEQ ID NO: 21)
CD34 Truncated
ATGGCCCTGCCTGTGACAGCCCTGCTGCTGCCTCTGGCTCTGCTGCTGCATGCCGCCA
GACCTGGATCTGAGGTGCAGCTGCAGCAGTCTGGCGCTGAACTCGTGCGGCCTG
GCGCTCTCGTGAAGCTGAGCTGTAAAGCCAGCGGCCTGAACATCAAGGACTACT
ACATGCACTGGGTCAAGCAGCGGCCCGAGCAGGGCCTGGAATGGATCGGCTGG
ATCGACCCCGAGAACGACAACACCAGATTCGACCCCCGGTTCCAGGGCCGGGT
GTCCATCATTGCCGACACCAGCAGCAACACCGCCTACCTGCAGCTGTCCAGCCT
GACCAGCGAGGACACCGCCGTGTACTATTGTGCCGCCGAGGCCTATTGGGGCC
AGGGCACACTCGTGACAGTGTCTGCTGGCGGCGGAGGATCTGGCGGAGGCGGAAG
TGGCGGGGGAGGAAGCGGAGGGGGCGGATCTGATATCGTGCTGACACAGAGCCCC
GCCTCCCTGGATGTGTCCCTGGGACAGAGAGCCACAATCAGCTGCAGAGCCTC
CCAGAGCGTGTCCACCAGCTCCTACAGCTATATGCACTGGTATCAGCAGAAGCC
CGGCCAGAGCCCCAAGCTGCTGATTAAGTACGCCAGCAACCTGGAAAGCGGCG
TGCCCGCCAGATTTTCTGGCAGCGGCTCTGGCACCGACTTCACCCTGAATATCC
ACCCCGTGGAAGAGGAAGATACCGCCACCTACTACTGCCAGCACAGCTGGGAG
ATCTGGACCTTCGGCGGAGGCACCAAGCTGGAAATCAAGtccggaaccacgacgccagcgcc
gcgaccaccaacaccggcgcccaccatcgcgtcgcagcccctgtccctgcgcccagaggcgtgccggccagcggcggggggcgc
agtgcacacgagggggctggacttcgcctgtgatatcTtt

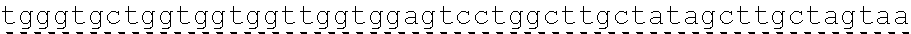

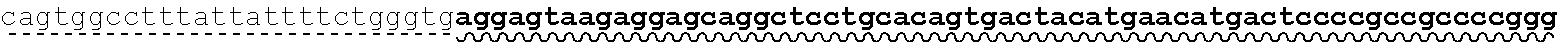

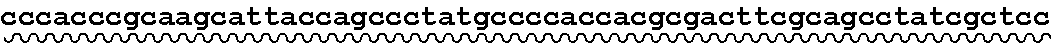

atcgatAGAGTGAAGTTCAG
CAGGAGCGCAGACGCCCCCGCGTACAAGCAGGGCCAGAACCAGCTCTATAACGAGC
TCAATCTAGGACGAAGAGAGGAGTACGATGTTTTGGACAAGAGACGTGGCCGGGAC
CCTGAGATGGGGGGAAAGCCGAGAAGGAAGAACCCTCAGGAAGGCCTGTACAATGA
ACTGCAGAAAGATAAGATGGCGGAGGCCTACAGTGAGATTGGGATGAAAGGCGAGC
GCCGGAGGGGCAAGGGGCACGATGGCCTTTACCAGGGTCTCAGTACAGCCACCAAG
GACACCTACGACGCCCTTCACATGCAGGCCCTGCCCCCTCGCGttaac 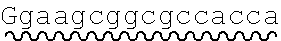 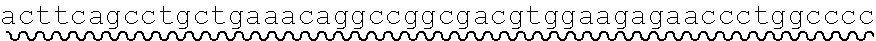
actagtgccaccatgctcgtgcggagaggcgccagagc
tggccccagaatgcctagaggatggaccgccctgtgcctgctgagcctgctgcctagcggcttcatgagcctggacaacaacggcaccgc
cacccctgagctgcctacccagggcaccttcagcaacgtgtccaccaatgtgtcctaccaggaaaccaccacccccagcaccctgggcag
cacatctctgcaccctgtgtcccagcacggcaacgaggaccaccaccaacatcaccgagacaaccgtgaagttcaccagcaccagcgtgat
cacctccgtgtacggcaacaccaacagcagcgtgcagagccagacctccgtgatcagcaccgtgtttaccacccccgctaatgtgtccacc
cccgaaaccaccctgaagcccagcctgtctcccggaaacgtgtccgacctgagcaccacctctaccagcctggccaccagccccaccaa
gccttacacaagcagcagccccatcctgagcgacatcaaggccgagatcaagtgcagcgcatccgggaagtgaagctgacacagggc
atctgcctggaacagaacaagaccagcagctgcgccgagttcaagaaggacagaggcgagggcctggccagagtgctgtgtggcgaa
gaacaggccgatgccgatgctggcgctcaagtgtgctctctgctgctggcccagagcgaagtgcggcctcagtgcctgctgctgtgctg
gccaacagaaccgagatcagcagcaaactgcagctgatgaagaagcaccagagcgacctgaagaagctgggcatcctggacttcaccg
agcaggacgtggcctcccaccagagctacagccagaaaaccctgatcgccctcgtgaccagcgagccctgctggcagtgctgaatc
accggctactttctgatgaaccggcggagctggtcccccaccggcgaaaga
Second generation α-synuclein targeting CAR construct (SEQ ID NO: 14):
Domains are labeled as follows:
(SEQ ID NO: 22)
CD8 Leader
(SEQ ID NO: 3)
aSyn (VH)
(SEQ ID NO: 23)
ScFv linker
(SEQ ID NO: 4)
aSyn (VL)
(SEQ ID NO: 24)
CD8 Hinge
(SEQ ID NO: 31)

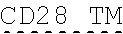

(SEQ ID NO: 9)

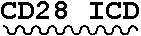

(SEQ ID NO: 26)
CD3z
(SEQ ID NO: 27)

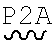

(SEQ ID NO: 28)
CD34 Truncated
MALPVTALLLPLALLLHAARPGSEVQLQQSGAELVRPGALVKLSCKASGLNIKDYYM
HWVKQRPEQGLEWIGWIDPENDNTRFDPRFQGRVSIIADTSSNTAYLQLSSLTSEDT
AVYYCAAEAYWGQGTLVTVSAGGGGSGGGGSGGGGSGGGGSDIVLTQSPASLDVSLG
QRATISCRASQSVSTSSYSYMHWYQQKPGQSPKLLIKYASNLESGVPARFSGSGSGT
DFTLNIHPVEEEDTATYYCQHSWEIWTFGGGTKLEIKSGTTTPAPRPPTPAPTIASQPLS
LRPEACRPAAGGAVHTRGLDFACDI 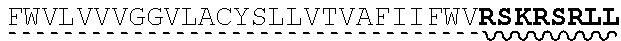 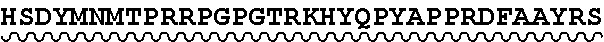
IDRVKFSRSADAPAYQQGQNQLYN
ELNLGRREEYDVLDKRRGRDPEMGGKPRRKNPQEGLYNELQKDKMAEAYSEIGMKGE
RRRGKGHDGLYOGLSTATKDTYDALHMOALPPRVN 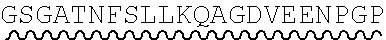
SATMLVRRGARAGPRMPRGWTALCLLSLLPSGFMSLDNNGTATPELPTQGTFSNVSTNV
SYQETTTPSTLGSTSLHPVSQHGNEATTNITETTVKFTSTSVITSVYGNTNSSVQSQTSVIS
TVFTTPANVSTPETTLKPSLSPGNVSDLSTTSTSLATSPTKPYTSSSPILSDIKAEIKCSGIRE
VKLTQGICLEQNKTSSCAEFKKDRGEGLARVLCGEEQADADAGAQVCSLLLAQSEVRPQ
CLLLVLANRTEISSKLQLMKKHQSDLKKLGILDFTEQDVASHQSYSQKTLIALVTSGALLA
VLGITGYFLMNRRSWSPTGER

### Introduction of Nucleic Acids

Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

RNA

In one embodiment, RNA is introduced into target cells. In another embodiment, the RNA is mRNA that comprises in vitro transcribed RNA or synthetic RNA. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is a chimeric membrane protein. By way of example, the template encodes an antibody, a fragment of an antibody or a portion of an antibody. By way of another example, the template comprises an extracellular domain comprising a single chain variable domain of an antibody, such as anti-CD3, and an intracellular domain of a co-stimulatory molecule. In one embodiment, the template for the RNA chimeric membrane protein encodes a chimeric membrane protein comprising an extracellular domain comprising an antigen binding domain derived from an antibody to a co-stimulatory molecule, and an intracellular domain derived from a portion of an intracellular domain of CD28 and 4-1BB.

PCR can be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary”, as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described elsewhere herein, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100 T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

In some embodiments, the RNA encoding bispecific minibodies is electroporated into the cells. In one embodiment, the RNA encoding bispecific minibodies is in vitro transcribed RNA.

The disclosed methods can be applied to the modulation of T cell activity in basic research and therapy, in the fields of cancer, stem cells, acute and chronic infections, and autoimmune diseases, including the assessment of the ability of the genetically modified T cell to kill a target cancer cell.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

One advantage of RNA transfection methods of the invention is that RNA transfection is essentially transient and a vector-free. A RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of T cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

RNA has several advantages over more traditional plasmid or viral approaches. Gene expression from an RNA source does not require transcription and the protein product is produced rapidly after the transfection. Further, since the RNA has to only gain access to the cytoplasm, rather than the nucleus, and therefore typical transfection methods result in an extremely high rate of transfection. In addition, plasmid based approaches require that the promoter driving the expression of the gene of interest be active in the cells under study.

In another aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

### Sources of T Cells

The methods described herein also include obtaining T cells from a subject. Non-limiting examples of subjects include humans, dogs, cats, mice, rats, and transgenic species thereof. Preferably, the subject is a human. T cells can be obtained from a number of sources, including peripheral blood mononuclear cells, bone marrow, lymph node tissue, spleen tissue, umbilical cord, and tumors. In certain embodiments, any number of T cell lines available in the art, may be used. In certain embodiments, T cells can be obtained from a unit of blood collected from a subject using any number of techniques known to the skilled artisan, such as Ficoll separation. In one embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood by lysing the red blood cells and depleting the monocytes, for example, by centrifugation through a PERCOLL™ gradient. Alternatively, T cells can be isolated from umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19 and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

In one embodiment, the purified population of T cells comprise regulatory T cells (Tregs). In one embodiment, the Tregs are obtained by isolating CD4+CD25+ T cells from a population of cells (e.g., peripheral blood mononuclear cells). In another embodiment, the Tregs are obtained by modifying a T cell to express FOXP3.

### Therapy

The modified T cells, e.g., modified Tregs, comprising a CAR as described herein (such as an α-syn CAR) may be included in a composition for therapy. The composition may include a pharmaceutical composition and further include a pharmaceutically acceptable carrier. A therapeutically effective amount of the pharmaceutical composition comprising the modified T cells may be administered.

Parkinson disease (PD) is the most common neurodegenerative disorder after Alzheimer disease (AD). Neuroinflammation is increasingly thought to be a contributor to Parkinson's Disease pathogenesis. alpha-synuclein fibrils can induce innate and adaptive immune responses and neuroinflammation can promote alpha-synuclein misfolding, creating a potential positive feedback loop for formation of the intra-neuronal aggregation of alpha-synuclein fibrils suggested to induce Parkinson's Disease pathology. T-cells from PD patients have been shown to recognize alpha-synuclein peptides and a variety of studies implicate T-cells and microglial activation in PD neurodegeneration. There are a number of clinical trials evaluating immunotherapies for PD. As described elsewhere herein, T-regulatory cells expressing an alpha-synuclein targeting CAR were generated with the aim of providing alpha-Synuclein fibril localized immunosuppression. Adoptive therapy with such alpha-synuclein CAR T-regs could potentially slow the progression of PD by dampening pathological microglial and glial activation and slowing the formation and spread of alpha-synuclein.

In one aspect, the invention includes a method for adoptive cell transfer therapy comprising administering a population of modified T cells capable of promoting α-synuclein specific immunosuppression in a subject. In one embodiment, the modified T cells further express a CAR, e.g., a CAR targeting α-synuclein. In one embodiment, the modified T cells are regulatory T cells (Tregs).

The modified T cells can be administered to an animal, preferably a mammal, even more preferably a human, to treat any disease known in the art to be related to α-synucleinopathy or α-synuclein aggregation. In some embodiments, the disease is selected from Parkinson's disease (PD), dementia with Lewy bodies (DLB, also known as Lewy body dementia), multiple system atrophy (MSA), and Alzheimer's disease with mixed Lewy pathology. In some embodiments, the disease is associated with an α-synucleinopathy. In some embodiments, the disease is a neurodegenerative disease.

In some embodiments, the disease is Parkinson's disease (PD). The treatment comprises administering to the subject a therapeutically effective amount of a pharmaceutical composition comprising a population of modified T cells capable of expressing a CAR.

In another embodiment, the T cells capable of expressing the CAR described herein may be used for the manufacture of a medicament for the treatment of a disease related to α-synuclein aggregation or synucleinopathies, e.g., Parkinson's disease, in a subject in need thereof.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

The cells of the invention to be administered may be autologous, allogeneic or xenogenic with respect to the subject undergoing therapy.

The administration of the cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient intracerebrally, transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

### Pharmaceutical Compositions

Pharmaceutical compositions of the present invention may comprise a modified T cell population (e.g., a modified Treg cell population) capable of expressing a CAR as described herein (such as an α-syn CAR), in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

It can generally be stated that a pharmaceutical composition comprising the modified T cells described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer modified T cells to a subject and then subsequently redraw blood (or have an apheresis performed), isolate T cells therefrom and further modify the T cells according to the present invention, and reinfuse the patient with these modified T cells. This process can be carried out multiple times every few weeks. In certain embodiments, T cells can be obtained from blood draws of from 10 ml to 400 ml. In certain embodiments, T cells are obtained from blood draws of 20 ml, 30 ml, 40 ml, 50 ml, 60 ml, 70 ml, 80 ml, 90 ml, or 100 ml. Not to be bound by theory, using this multiple blood draw/multiple reinfusion protocol, may select out certain populations of T cells.

In certain embodiments of the present invention, T cells are administered to a patient in conjunction with (e.g., before, simultaneously or following) any number of relevant treatment modalities.

The dosage of the treatments to be administered to a patient will vary with the precise nature of the condition being treated and the recipient of the treatment. The scaling of dosages for human administration can be performed according to art-accepted practices. The dose for CAMPATH antibody, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook et al., 2012, volumes 1-4, Cold Spring Harbor Laboratory Press, NY); “Oligonucleotide Synthesis” (M. J. Gait, ed., Oxford University Press, 1984); “Culture of Animal Cells” (R. I. Freshney, Wiley-Blackwell, 2010); “Methods in Enzymology” (S. P. Colowick, N. O. Kaplan, et al., eds., volumes 1-650, Academic Press); “Handbook of Experimental Immunology” (D. M. Weir et al., Wiley, 1997); “Gene Transfer Vectors for Mammalian Cells” (J. Miller and M. P. Calos, Cold Spring Harbor Laboratory Press, N Y, 1987); “Short Protocols in Molecular Biology” (F. M. Ausubel et al., eds., John Wiley & Sons, 2002); “Polymerase Chain Reaction: Principles, Applications and Troubleshooting”, (M. E. Babar, VDM Verlag Dr. Muller, 2011); “Current Protocols in Immunology” (J. E. Coligan et al., eds., John Wiley & Sons, 1991). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

Parkinson Disease (PD) is a common progressive neurodegenerative disorder in which neuron loss in the substantia nigra (SN) leads to the clinical sequelae of dopamine deficiency. The pathological hallmark of the disease, intra-neuronal aggregation of α-synuclein fibrils into Lewy bodies, has been recognized for over a century, but the complexity of the underlying and resultant molecular pathogenesis remains incompletely understood. Regardless of the initial genetic or toxic trigger, neuroinflammation is increasingly thought to be a necessary contributor to PD pathogenesis (Ransohoff, Science, 2016 Aug. 19; 353(6301):777-83; Hirsch et al., Lancet Neurol. 2009 April; 8(4):382-97). Whereas α-synuclein aggregation can induce innate and adaptive immune responses, neuroinflammation can in turn promote α-synuclein misfolding, thereby feeding a self-reinforcing cycle (Gao et al., J. Neurosci. 2008 Jul. 23; 28(30):7687-98; Tome et al., Mol. Neurobiol. 2013 April; 47(2):561-74).

There is preclinical evidence that α-synuclein can induce PD pathology and that pathology can be prevented by blocking transmission of α-synuclein (Luk et al., Science, 2012 Nov. 16; 338(6109):949-53; Tran et al., Cell Rep. 2014 Jun. 26; 7(6):2054-65; Spencer et al., Acta Neuropathol Commun. 2017 Jan. 13; 5(1):7; El-Agnaf et al., Neurobiol Dis. 2017 August; 104:85-96). T cells from patients with PD have been shown to recognize α-synuclein peptides, perhaps explaining the association of PD risk with certain major histocompatibility complex alleles (Sulzer et al., Nature, 2017 Jun. 28; 546(7660):656-661; Hamza et al., Nat Genet. 2010 September; 42(9):781-5). Furthermore, α-synuclein reactive T cells may be found years before the diagnosis of PD (Arlehamn et al., Nat Commun. 2020 Apr. 20; 11(1):1875). A variety of animal studies implicate T cells and microglial activation in PD neurodegeneration, and tie these to overexpression of α-synuclein (Brochard et al., J Cin Invest. 2009 January; 119(1):182-92; Cebrian et al., Curr Top Behav Neurosci. 2015; 22:237-70; Iba et al., J Neuroinflammation. 2020 Jul. 17; 17(1):214; Theodore et al., J. Neuropathol Exp Neurol. 2008 December; 67(12):1149-58). Interest in immunotherapy for PD has manifested in a number of clinical trials including vaccines, small molecules and monoclonal antibodies (Zella et al., Neurol Ther. 2019 June; 8(1):28-44).

In the study described herein, it was hypothesized that α-synuclein-specific immunosuppression could halt the progression of PD by inhibiting the spread of α-synuclein while dampening pathologic microglial and glial activation. Regulatory T cells have the potential to exert antigen-specific immunosuppression of a variety of immune cells.

Adoptive immunotherapy with Tregs is of great interest in the treatment of immune-mediated diseases (Raffin et al., Nat Rev Immunol. 2020 March; 20(3):158-172). Human regulatory T cells (Tregs) can be enriched by genetic expression in CD4 cells of the canonical Treg transcription factor FOXP3, or by sorting CD4+CD127-CD25++ T cells followed by ex vivo expansion. However, the resultant polyclonal Tregs are less potent than antigen-specific Tregs, leading to current interest in the development of genetically engineered Tregs of defined specificity (Mikami et al., Curr Opin Immunol. 2020 Aug. 19; 67:36-41). Tregs bearing chimeric antigen receptors or transgenic T cell receptors have been developed for auto- or alloimmune indications by several groups, and the optimal costimulatory molecules for use in genetically engineered human T cells were recently published (Dawson et al., Sci Transl Med. 2020 Aug. 19; 12(557):eaaz3866; Boroughs et al., JCI Insight, 2019 Mar. 14; 5(8):e126194; Hull et al., J Autoimmun. 2017 May; 79:63-73; Seng et al., Blood Adv. 2020 Apr. 14; 4(7):1325-1339).

PD remains incurable despite the development of multiple therapeutic paradigms, including palliative dopamine replacement, deep brain stimulation gene therapy and regenerative medicine approaches. The study herein contributes to deepening understanding of PD immunopathogenesis and advances in genetically engineered adoptive cell therapy by designing and testing anti-α-synuclein regulatory T cells to prevent the progression of PD.

The materials and methods employed in these experiments are now described.

Cloning of Lentiviral Products and Lentiviral Packaging

The anti-alpha synuclein variable fragment genetic sequence was obtained from the Luk lab. This was digested with SalI-HF (New England Biolabs) and BspEI (New England Biolabs) and ligated into a third-generation replication-deficient lentiviral vector containing the bicistronic cassette of a CD3ζ T-cell stimulatory domain and a functionally deficient CD34 tag connected downstream by a P2A linker region. A CD28-CD3ζ CAR construct was PCR amplified to introduce the BspEI and HpaI (New England Biolabs) restriction sites. The PCR product was digested and ligated in to generate the second-generation CAR construct. The same process was followed to generate the αCD19 CAR. All CAR constructs were driven by an EF1α promoter.

Lentiviral Production

Replication-deficient lentivirus was produced using HEK293T cells (ATCC, CRL-3216) as previously described. The day before transfection, cells were washed with DPBS (Corning) and trypsinized with 0.05% Trypsin (Gibco). 8×10⁶-1×10⁷ 293T cells were plated on a 150 cm² flask in 30 mL of DMEM (10-013-CM) supplemented with HEPES (Gibco), Penicillin/Streptomycin (BioWhittaker), FBS (SeraDigm), and Glutamax (Gibco). One day after plating, cells were transfected with 18 g pRSV.Rev, 18 μg pMDLg/p.RPE, 7 g pVSV-G, and 15 μg transfer plasmid in OptiMem with lipofectamine 2000 overnight.

Cell Culture

CD4-selected T-cells were ordered from the Human Immunology Core and allowed to rest overnight. These cells were then stained with APC αCD25 (Invitrogen) and enriched for CD25 using aAPC magnetic beads (Miltenyi) per manufacturer's instructions. The CD25 enriched population was stained with APC eFluor 780 αCD127 (Invitrogen), PE αCD45RA (Biolegend), and Pacific Blue αCD4 (Biolegend) and sorted at the Wistar Flow Cytometry Core for the CD4+CD45RA++CD127-CD25+++ population. These cells were resuspended in CTS Optimizer media (ThermoFisher) supplemented with 2% human serum (Gemini), 100 U/mL Penicillin/Streptomycin, and 1× GlutaMax to a concentration of 5×10⁵ cells/mL and activated with 3-4× as many CD3/CD28 activation beads (Gibco). Treg media was supplemented with 300 IU/mL IL-2 (Peprotech) and Tconv media was supplemented with 20 IU/mL IL-2 for expansion. IL-2 was supplemented every 2-3 days, and cell volume was assessed by Coulter Counter starting at day 5 until the end of the expansion. Post-expansion, CAR identity and Treg identity were assessed using BV605 αCD4 (Biolegend), PE αFoxP3 (Biolegend), APC αCD34 (Biolegend), AlexaFluor 488 αCD25 (Biolegend), and Live/Dead Fixable Violet Dead Cell Stain (ThermoFisher). For intracellular staining, cells were fixed and permeabilized using a FoxP3 staining kit (Invitrogen). Samples were run on a BD LSR Fortessa, and all data were analyzed using the FlowJo software.

Activation Assay

Ninety six-well flat-bottom plates (Falcon) were conditioned with 100 μL of alphasynuclein pre-formed fibrils (PFFs) at 10 ng/mL or vehicle (DPBS) for 24 hours prior to the onset of the assay. 100 μL of cells were then introduced at a concentration of 1×10⁶ cells/mL and allowed to co-culture with the PFFs, 4×10⁵ CD3/CD28 activation beads, or complete media alone for 24 hours at 37° C., 5% CO2. Following coculture, cells were moved to a 96-well round bottom plate (Denville) and centrifuged at 1000×g for 3 minutes at RT to remove cells from solution. The supernatant was then removed and either used fresh or frozen at −80° C. before use. Cells were stained using PerCP eFluor 710 αCD39 (Invitrogen), FITC αCD69 (Biolegend), PE αLAP (Invitrogen), and BV421 αCTLA-4 (Biolegend) antibodies to assess activation and Treg identity by flow cytometry. Samples were run on a BD LsrFortessa, and all data were analyzed by FlowJo.

Proliferation Assay

Cells were used fresh or frozen after overnight rest in complete media. Conventional second generation anti-α synuclein T cells were stained using cell trace violet (Invitrogen, C34557A). 5×10⁴ Tconv were plated in 200 μL of complete media at cellular ratios of 4:1, 2:1, 1:1, or 0:1 (Treg:Tconv) in flat-bottom 96-well plates that had been conditioned with 100 μL of PBS with 10 ng/mL of a synuclein PFFs. Triplicate wells of non-stimulated Tconvs were plated to establish a non-proliferative peak. Media was removed to assess cytokines at 24 h and 120 h. Cells were centrifuged at 1000×g for 3 minutes at RT, and stained with BV605 αCD4 and APC αCD8 (Biolegend) before being analyzed by flow cytometry. Data was analyzed using FlowJo.

Cytokine Bead Array

Supernatants from the activation and proliferation assays were analyzed using the Th1/Th2/TH17 cytokine bead array (BD) per manufacturer's instructions. Samples were run on an LSR Fortessa flow cytometer. Data was analyzed using FCAP array 3.0.

In Vivo Studies

NOD-scid IL2Rγ^{null} mice were purchased from the Stem Cell and Xenograft Core (University of Pennsylvania). 6-12 week old mice were housed in the University of Pennsylvania Institutional Animal Care & Use Committee approved facilities. Mice were injected intracerebrally with a synuclein PFFs at 15 μg/animal. Injections were targeted to 3 different brain regions (ventral striatum, dorsal striatum, and overlaying cortex) as described previously (Ugras et al., EBioMedicine. 2018 May; 31:307-319). At 6-8 weeks post injection, mice were injected with second generation a synuclein targeting Treg, second-generation α synuclein targeting Tconv, second generation CAR19 Tregs, second-generation CAR19 Tconv, or mixtures of these cells.

The results of the experiments are now described.

### Example 1: Targeting Parkinson's Disease (PD) with Engineered Regulatory T Cells

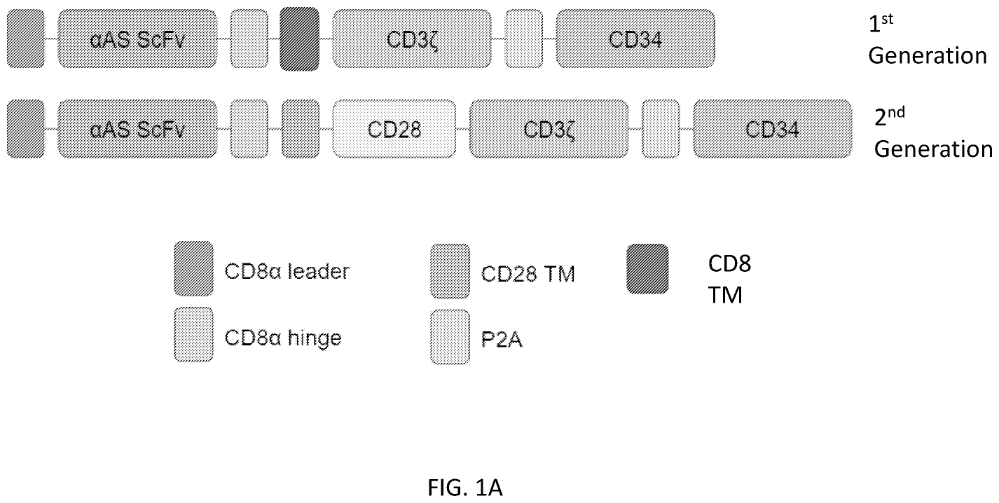

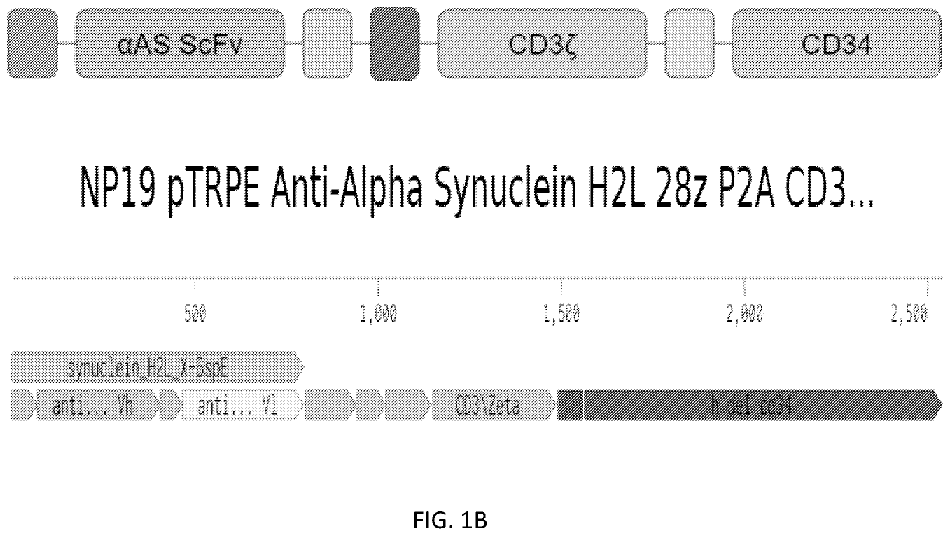

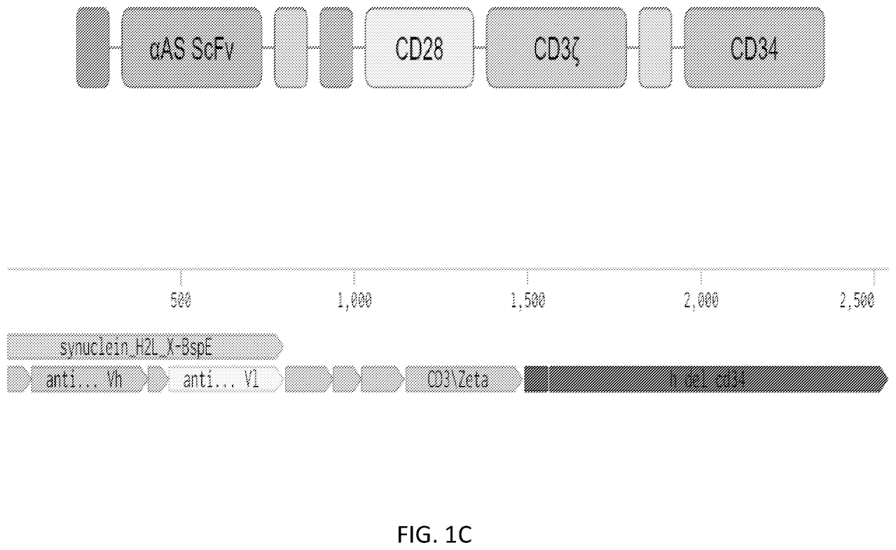

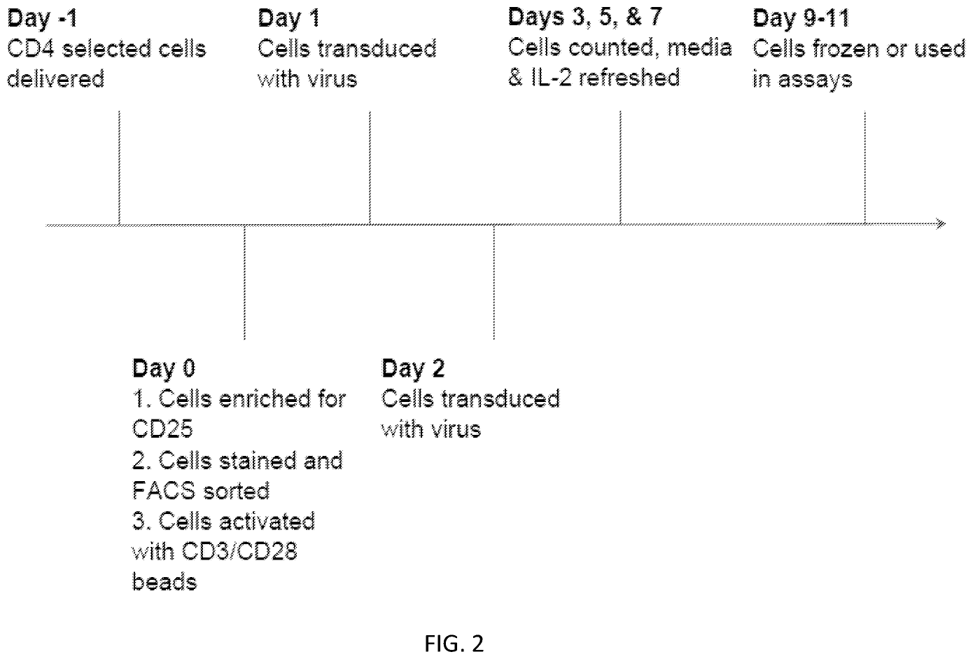

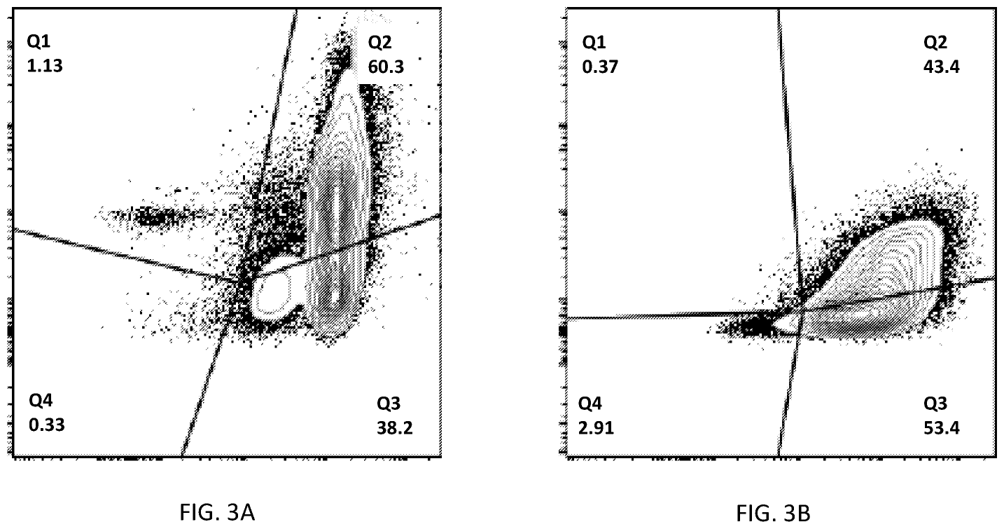

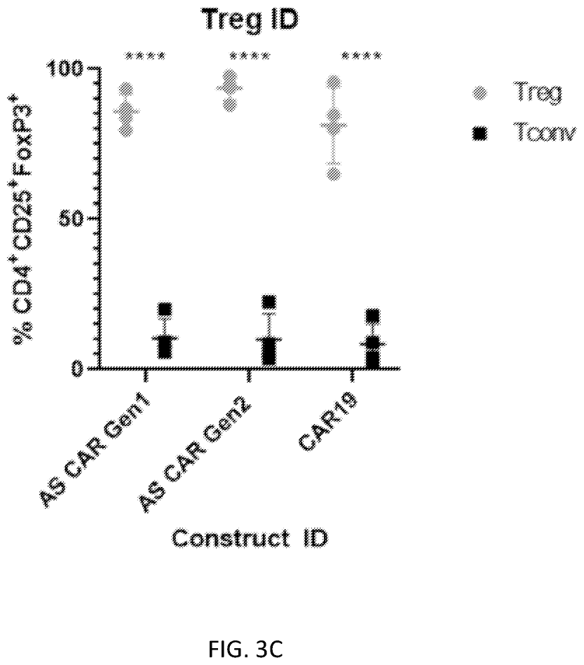

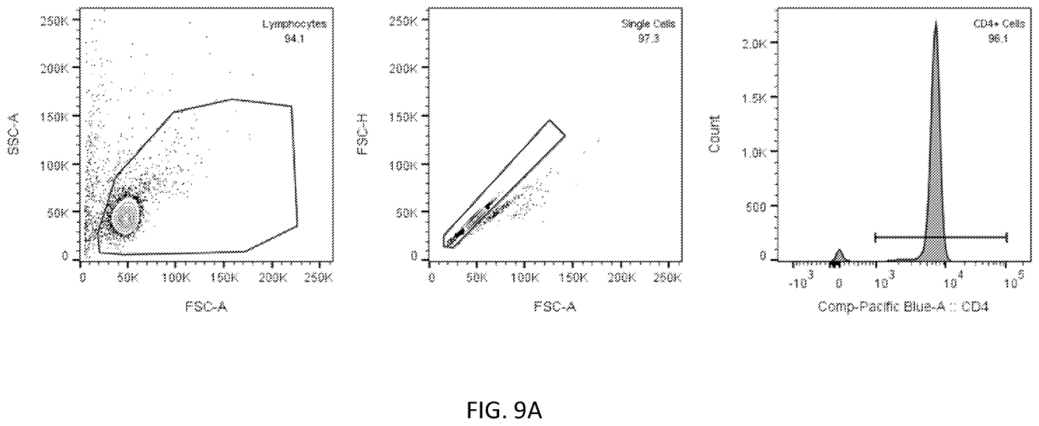

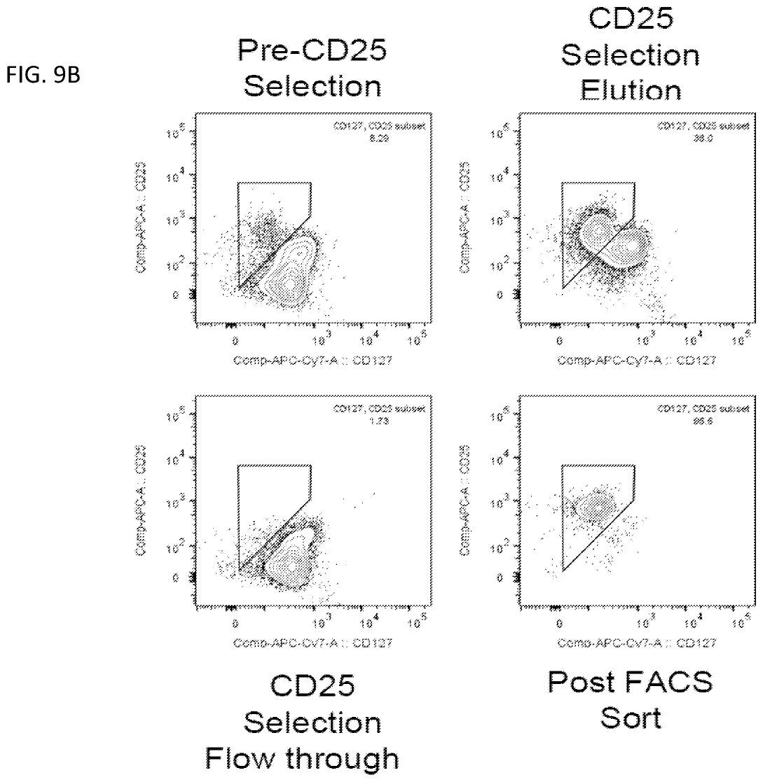

Regulatory or conventional effector T cells directed against α-synuclein were made by cloning the sequence of the light and heavy chains of an anti-α-synuclein monoclonal antibody (Syn303) recognizing the N-terminal epitope of pathological α-synuclein into the lentiviral pTRPE chimeric antigen receptor backbone (Tran et al., Cell Rep. 2014 Jun. 26; 7(6):2054-65). Based on recent reports that CD28-costimulated CAR Tregs mediate more potent immunosuppression than those costimulated with 4-1BB, anti-α-synuclein CAR T regs (nASCAR Tregs) bearing CD28 (“second generation CAR”) or lacking costimulatory domains (“first generation CAR”) were made. As controls, anti-CD19 CAR T cells with the same configurations were made. A truncated domain from human CD34 was used as a reporter (FIGS. 1A-1C). The position of the VH and VL domains of the AS-binding domain in the first and second generation α-synuclein CAR's are indicated in FIG. 1B and FIG. 1C, respectively. The full nucleic acid sequences of the first and second generation α-synuclein CAR's are indicated in SEQ ID NO: 11 and SEQ ID NO: 13, respectively. The full amino acid sequences of the first and second generation α-synuclein CAR's are indicated in SEQ ID NO: 12 and SEQ ID NO: 14, respectively. Tregs were made by magnetic enrichment of human CD4+ T cells for CD25, followed by flow sorting of CD4+CD25++ population, followed by activation with anti-CD3/CD28 Dynabeads, lentiviral transduction and expansion in media supplemented with interleukin-2 (FIG. 2 and FIG. 9A-9B). Transduction was confirmed by staining for CD34, (FIG. 3A) and the Treg phenotype was confirmed by staining for CD4, FOXP3 and CD25 (FIG. 3B). Quantitation of the markers in Treg and Tconv cells transduced with the three CAR constructs is shown in FIG. 3C.

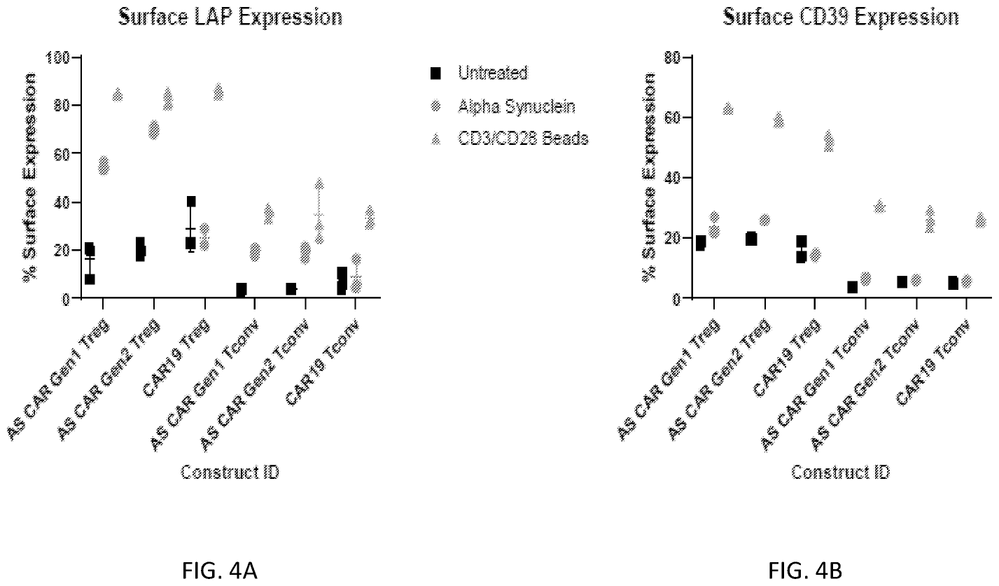

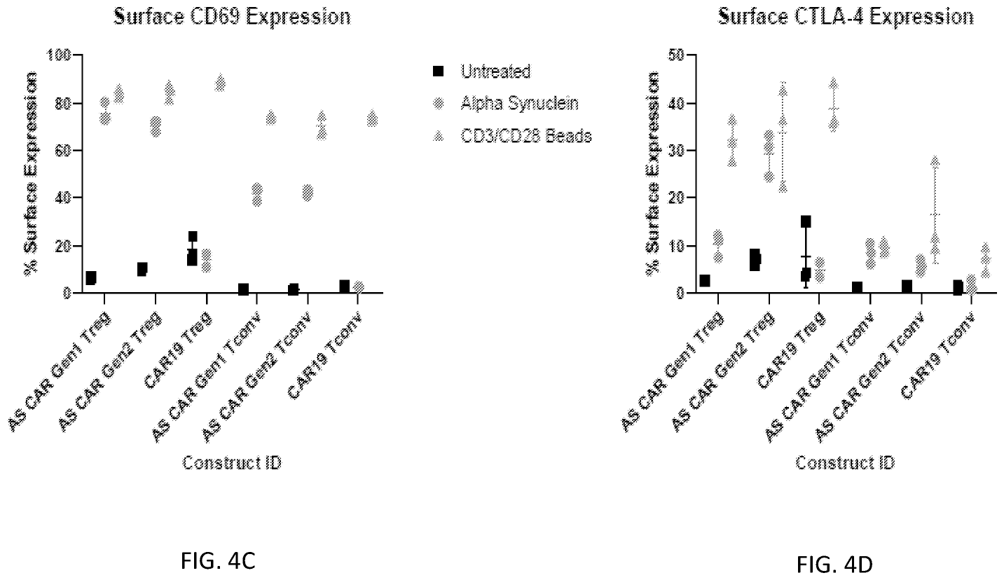

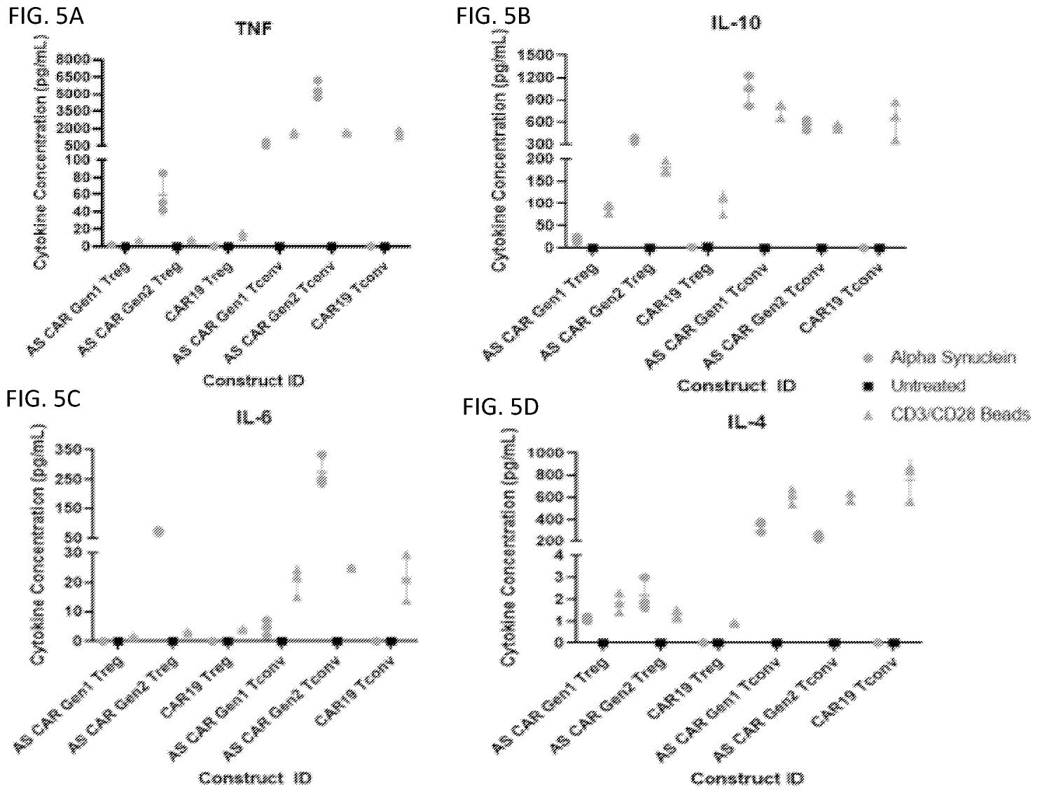

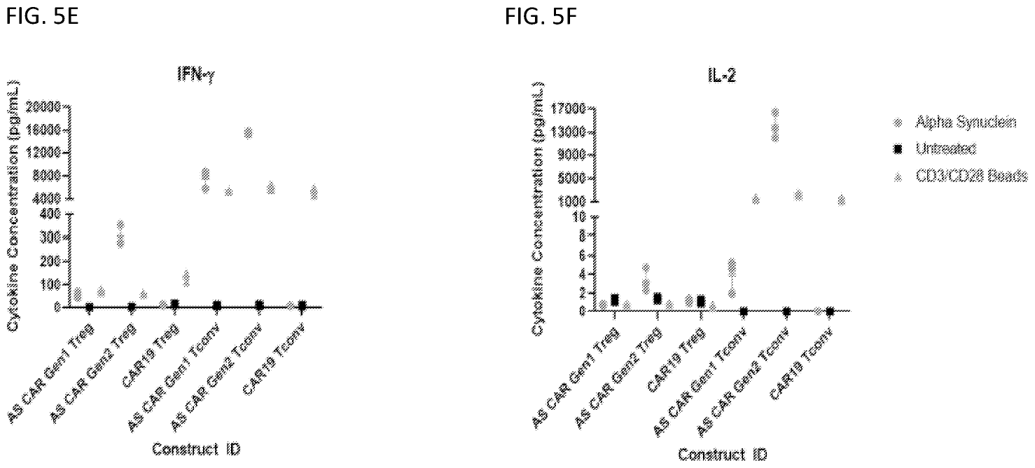

To test the function of nASCAR T cells, the cells were stimulated in vitro with preformed α-synuclein fibrils (PFF) or with anti-CD3/CD28 beads as a positive control. In comparison to nASCAR or CD19-specific Tconv, unstimulated nASCAR and CD19-specific Tregs exhibited higher expression of the Treg-specific molecules Latency Associated Peptide (LAP), CD39 and surface CTLA4 at baseline (Chen et al., J Immunol. 2008 Jun. 1; 180(11):7327-37; Mandapathil et al., J Biol Chem. 2010 Mar. 5; 285(10):7176-86; Manuszak et al., J Biol Methods. 2020 Apr. 13; 7(2):e131). Unspecific stimulation with anti-CD3/CD28 beads led to further increase in expression of LAP, CD39 and CTLA4 as well as CD69, consistent with activation. Specific stimulation with α-synuclein PFF led to upregulation of LAP and CD69 in first- and second-generation nASCAR Tregs, while surface CTLA4 upregulation was specific to second-generation nASCAR Tregs (FIG. 4A-4D). Tconv nASCAR produced IL-2, IL-4, IFN-γ, and TNF. While nASCAR Tregs produced generally lower amounts of cytokines, second generation nASCAR Tregs specifically produced the immunosuppressive cytokine IL-10 in response to α-synuclein (FIGS. 5A-5F).

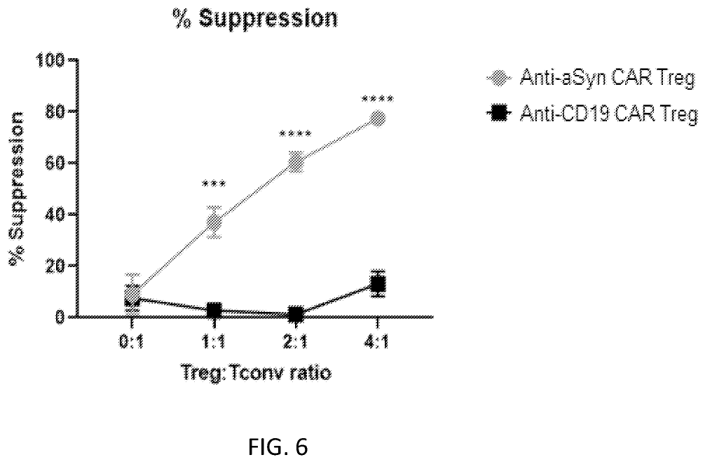

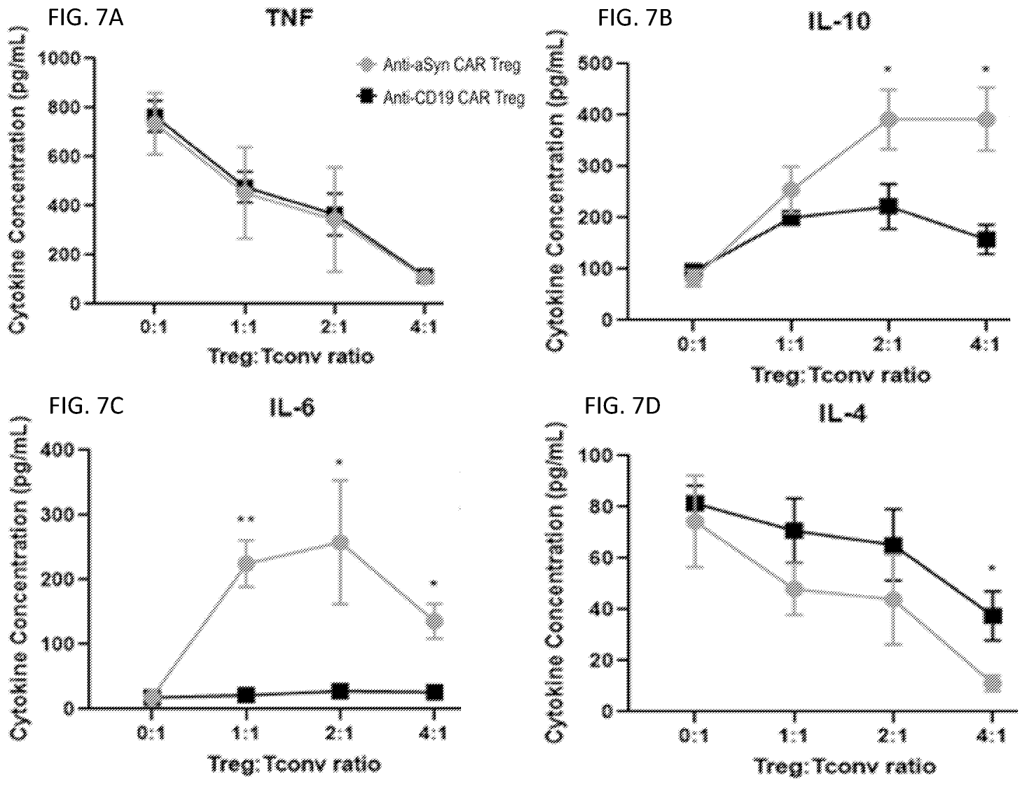

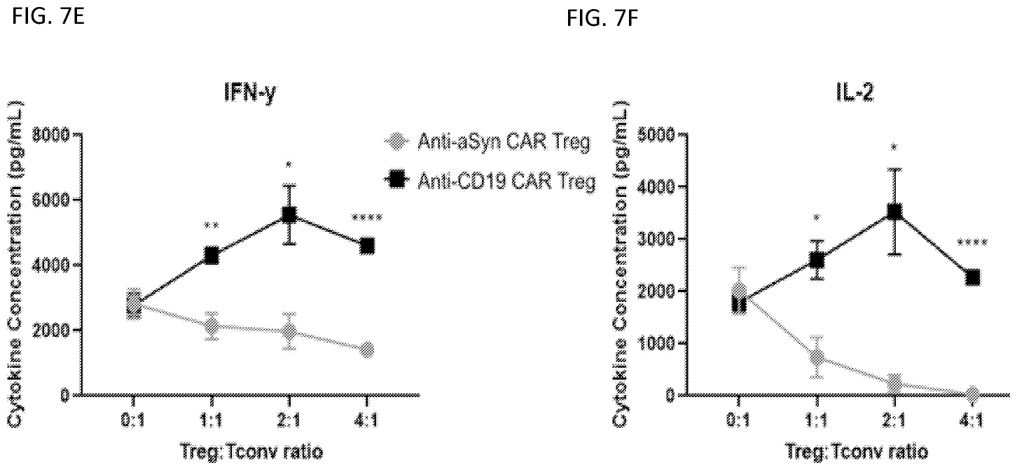

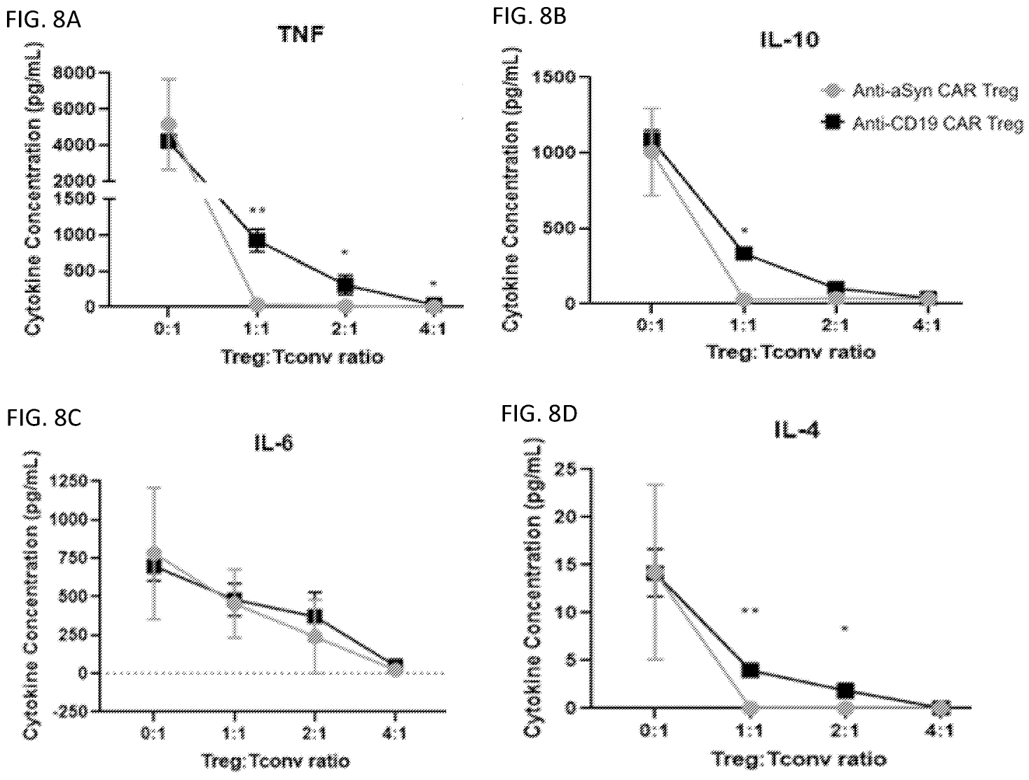

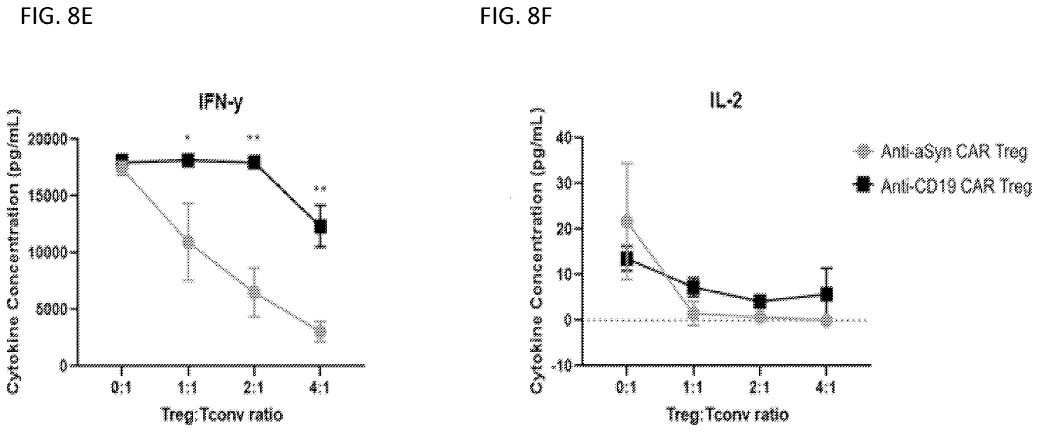

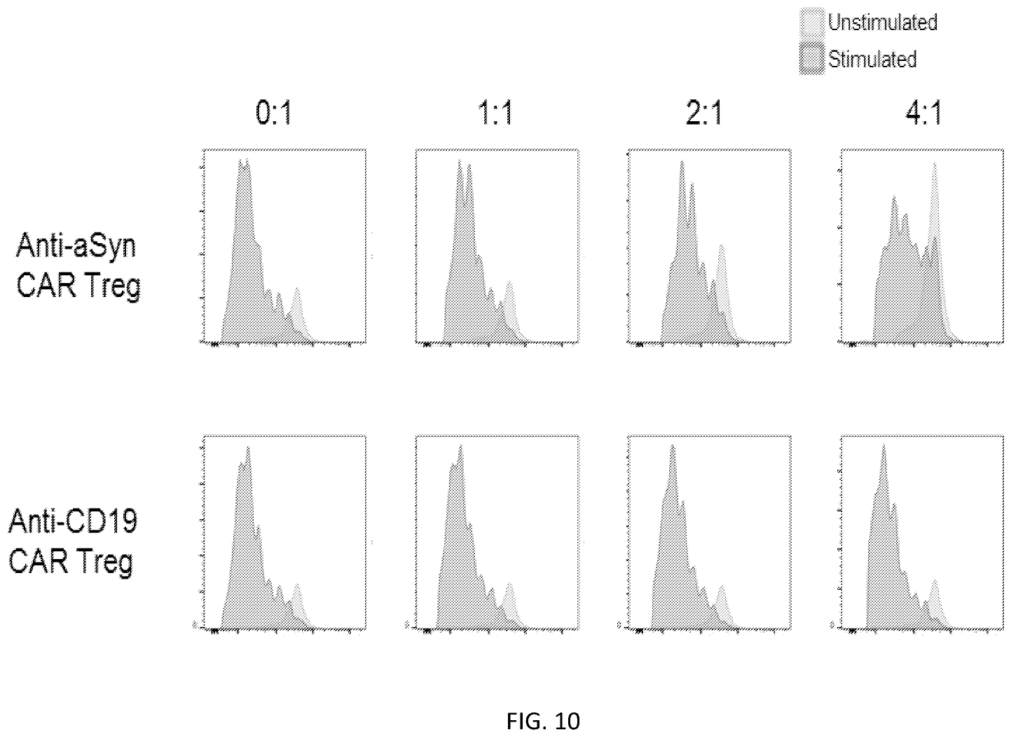

Next, the study sought to determine whether nASCAR Tregs could block the proliferation and activation of effector nASCAR Tconv in response to α-synuclein. Using a classical suppression assay, it was found that nASCAR Tconv proliferated extensively upon exposure to α-synuclein PFF, and that this was inhibited in the presence of antigen-specific nASCAR Tregs but not in the presence of control anti-CD19 CAR Tregs (FIG. 6 and FIG. 10). Antigen-specific nASCAR Tregs were also able to block the production of IL-2, IL-6, IL-10 and IFNγ at 24 and 120 hours (FIGS. 7A-7F and FIGS. 8A-8F, respectively).

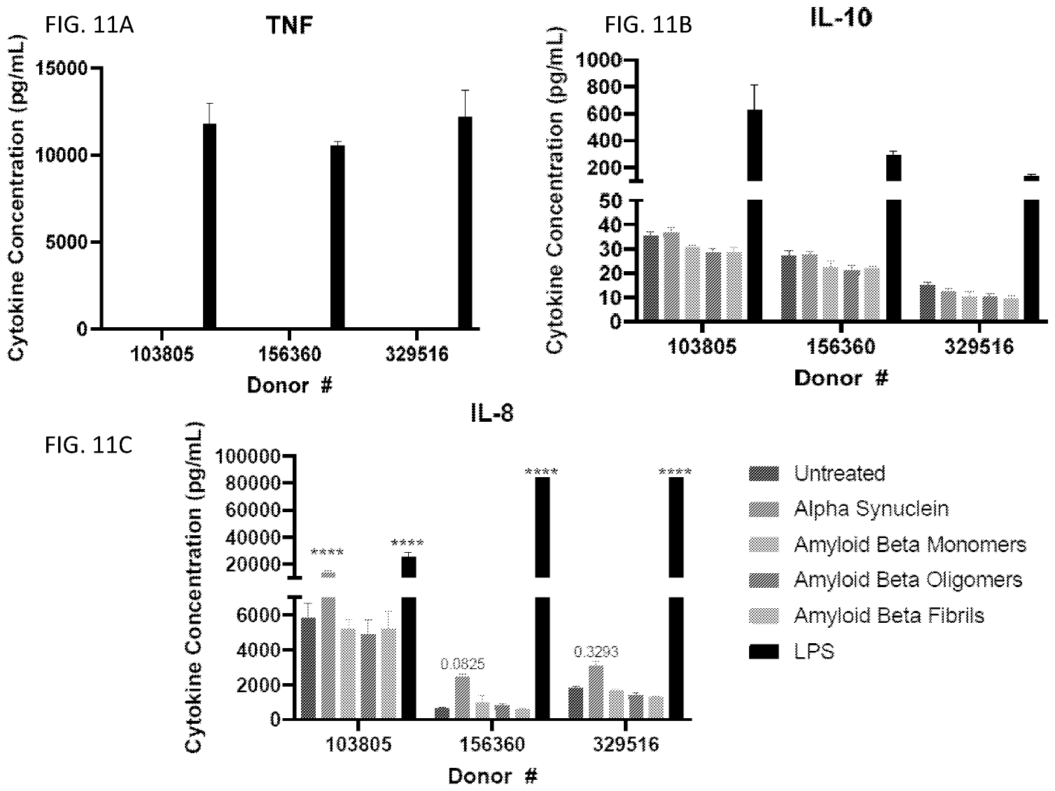

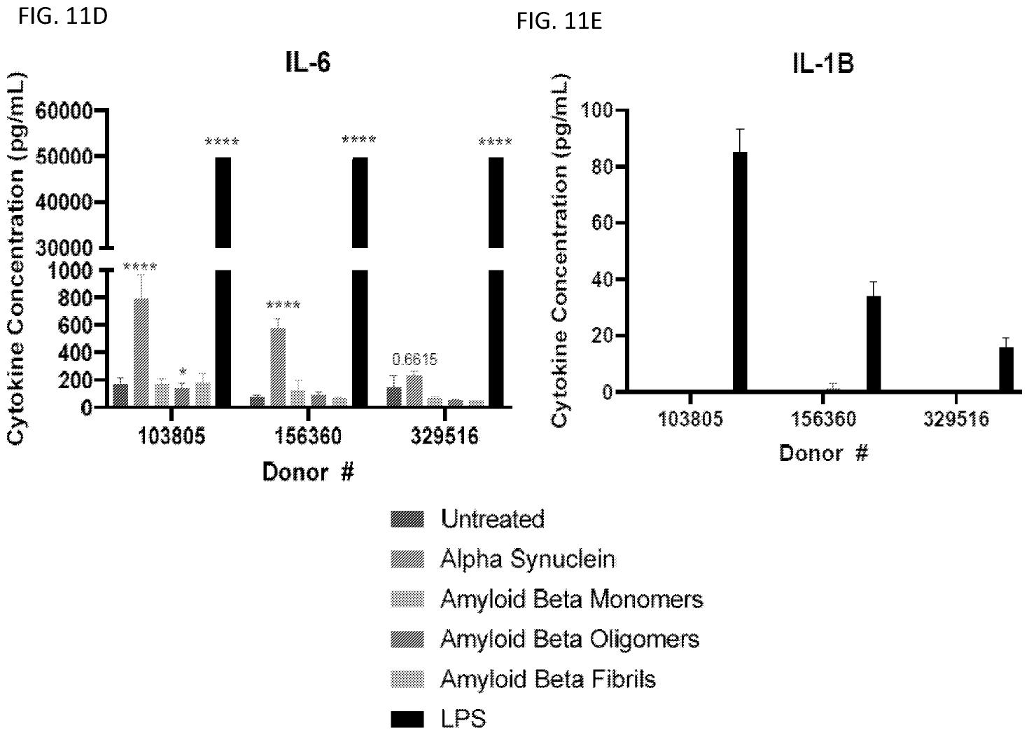

Emerging data indicate a central role for microglia in PD immunopathology. The impact on primary human macrophages of exposure to α-synuclein PFF was first determined by measuring cytokine production (FIGS. 11A-11E). The impact on primary human macrophages of exposure to α-synuclein PFF is also determined by measuring transcriptional changes. Cytokine production and transcriptional changes in primary human macrophages exposed to α-synuclein PFF with the addition of nASCAR Tregs are also measured.

Next, primary human neurons are cultured with α-synuclein PFF along with nASCARTregs or nASCARTconv. Effector T cells mediate destruction of α-synuclein-expressing neurons, a pathologic effect that is partially rescued by addition of nASCAR Tregs.

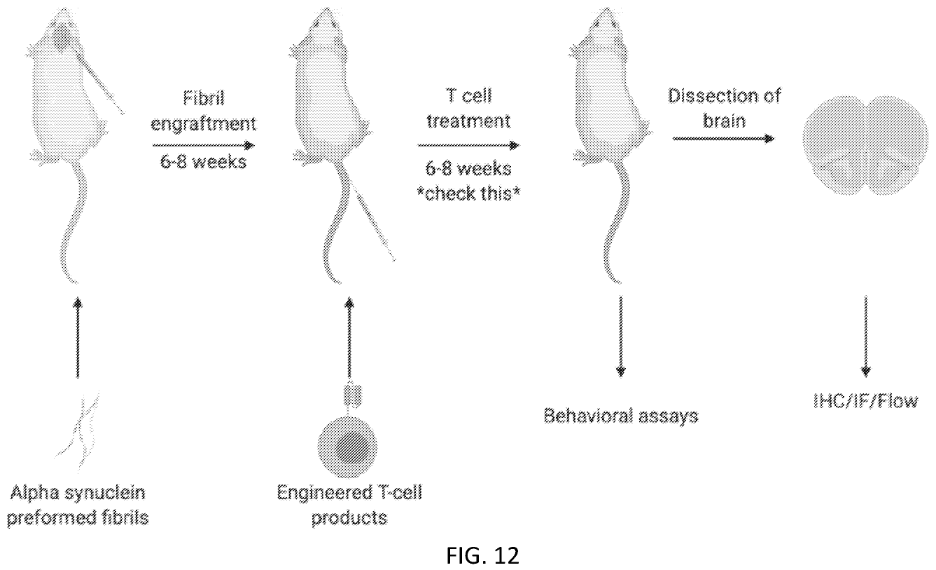

Finally, immunodeficient NSG mice were injected with α-synuclein PFF followed by nASCAR or control T cells given intravenously (FIG. 12). At 6-8 weeks following T cell injection, the mice were euthanized and brains were removed for microscopic analysis of α-synuclein spread, neuronal pathology (tyrosine hydroxylase staining) and T cell infiltration. A single infusion of nASCAR Tregs mediates reduction of α-synuclein spread, prevents dopaminergic neuron loss and is accompanied by other changes.

### DISCUSSION

After years of preclinical and clinical development, CART cells are now available by a physician's prescription for some patients with B-cell malignancies, and many more are in development for other malignancies. The efficacy and safety of genetically engineered T cells in cancer are now well known, and the field is turning its attention to other innovative applications of this modality. Preclinical models have recently shown that CAR T cells can be used to reverse cardiac fibrosis and cellular senescence (Aghajanian et al., Nature, 2019 September; 573(7774):430-433; Amor et al., Nature, 2020 July; 583(7814):127-132), diseases in which T cells were not previously thought to play a key role.

Tregs can mediate broad immunosuppressive effects in an antigen-specific manner, and genetically engineered CAR T regs are being developed in the setting of auto- or allo-immune diseases. While the relative contribution of the immune system in PD pathogenesis remains controversial, a growing body of evidence now points to T cell- and microglia-mediated neuroinflammation as being necessary, if not sufficient, for disease progression.

To bring together the two disparate streams of innovation, α-synuclein-specific human CAR Tregs (nASCAR Tregs) were developed in the study herein, and results of the study showed that they inhibit α-synuclein-mediated effector T cell and macrophage-based inflammation. These results provide a proof-of-concept for the application of a CAR Treg based cellular immunotherapy for the treatment of PD.

Despite these hurdles, nASCAR Tregs may offer an exciting new avenue for PD treatment. Cellular immunotherapy has revolutionized the cancer treatment by allowing a refined, targetable approach in place of chemotherapy. With further refining, CAR Tregs will likely provide one of the first curative treatment options for patients with PD.

### Other Embodiments

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An isolated nucleic acid encoding a chimeric antigen receptor (CAR), the CAR comprising an antigen binding domain, a transmembrane domain, and an intracellular domain, wherein the antigen binding domain specifically binds to α-synuclein.

2. The isolated nucleic acid of claim 1, wherein the antigen binding domain comprises a heavy chain variable (VH) domain encoded by a nucleic acid sequence at least 85%, 90%, 95%, 96%, 97%, 98%, 99%, or 100% identical to SEQ ID NO: 1, and/or a light chain variable domain (VL) domain encoded by a nucleic acid sequence at least 85%, 90%, 95%, 96%, 97%, 98%, 99%, or 100% identical to SEQ ID NO: 2.

3. The isolated nucleic acid of claim 1, wherein the intracellular domain comprises a signaling domain and/or an intracellular domain of a costimulatory molecule.

4. The isolated nucleic acid of claim 3, wherein the signaling domain is a CD3 zeta signaling domain.

5. The isolated nucleic acid of claim 3, wherein the costimulatory molecule is CD28.

6. The isolated nucleic acid of claim 3, wherein the intracellular domain comprises a CD3 zeta signaling domain and a CD28 costimulatory molecule.

7. The isolated nucleic acid of claim 1, wherein the CAR is encoded by a nucleic acid sequence at least 85%, 90%, 95%, 96%, 97%, 98%, 99%, or 100% identical to SEQ ID NO: 11 or SEQ ID NO: 13.

8. A chimeric antigen receptor (CAR) comprising an antigen binding domain, a transmembrane domain, and an intracellular domain, wherein the antigen binding domain specifically binds to α-synuclein.

9. The CAR of claim 8, wherein the antigen binding domain comprises a heavy chain variable (VH) domain comprising HCDR3, HCDR2, and HCDR1 regions, and a light chain variable (VL) domain comprising LCDR3, LCDR2 and LCDR1 regions, wherein
the HCDR3 region comprises the amino acid sequence AAEAY (SEQ ID NO: 5) and/or;
the HCDR2 region comprises the amino acid sequence IDPENDNT (SEQ ID NO: 6) and/or;
the HCDR1 region comprises the amino acid sequence GLNIKDYY (SEQ ID NO: 7) and/or;
the LCDR3 region comprises the amino acid sequence QHSWEIWT (SEQ ID NO: 8) and/or;
the LCDR2 region comprises the amino acid sequence YAS and/or;
the LCDR1 region comprises the amino acid sequence QSVSTSSYSY (SEQ ID NO: 10).

10. The CAR of claim 8, wherein the antigen binding domain comprises a heavy chain variable (VH) domain comprising an amino acid sequence least 85%, 90%, 95%, 96%, 97%, 98%, 99%, or 100% identical to SEQ ID NO: 3, and/or a light chain variable domain (VH) domain comprising an amino acid sequence least 85%, 90%, 95%, 96%, 97%, 98%, 99%, or 100% identical to SEQ ID NO: 4.

11. The CAR of claim 8, wherein the antigen binding domain comprises a heavy chain variable (VH) domain encoded by a nucleic acid sequence at least 85%, 90%, 95%, 96%, 97%, 98%, 99%, or 100% identical to SEQ ID NO: 1, and/or a light chain variable domain (VH) domain encoded by a nucleic acid sequence at least 85%, 90%, 95%, 96%, 97%, 98%, 99%, or 100% identical to SEQ ID NO: 2.

12. The CAR of claim 8, wherein the intracellular domain comprises a signaling domain and/or an intracellular domain of a costimulatory molecule.

13. The CAR of claim 12, wherein the signaling domain is a CD3 zeta signaling domain.

14. The CAR of claim 12, wherein the costimulatory molecule is CD28.

15. The CAR of claim 12, wherein the intracellular domain comprises a CD3 zeta signaling domain and a CD28 costimulatory molecule.

16. The CAR of claim 8, wherein the CAR comprises an amino acid sequence at least 85%, 90%, 95%, 96%, 97%, 98%, 99%, or 100% identical to SEQ ID NO: 12 or SEQ ID NO: 14.

17. The CAR of claim 8, wherein the CAR is encoded by a nucleic acid sequence at least 85%, 90%, 95%, 96%, 97%, 98%, 99%, or 100% identical to SEQ ID NO: 11 or SEQ ID NO: 13.

18. A genetically modified cell comprising the CAR of claim 8.

19. The cell of claim 17, wherein the cell is a regulatory T cell (Treg).

20. A genetically modified regulatory T cell comprising a CAR, wherein the CAR comprises:
i) an antigen binding domain comprising a heavy chain variable (VH) domain comprising HCDR3, HCDR2, and HCDR1 regions, and a light chain variable (VL) domain comprising LCDR3, LCDR2 and LCDR1 regions, wherein
the HCDR3 region comprises the amino acid sequence AAEAY (SEQ ID NO: 5) and/or;
the HCDR2 region comprises the amino acid sequence IDPENDNT (SEQ ID NO: 6) and/or;
the HCDR1 region comprises the amino acid sequence GLNIKDYY (SEQ ID NO: 7) and/or;
the LCDR3 region comprises the amino acid sequence QHSWEIWT (SEQ ID NO: 8) and/or;
the LCDR2 region comprises the amino acid sequence YAS and/or;
the LCDR1 region comprises the amino acid sequence QSVSTSSYSY (SEQ ID NO: 10); and
ii) an intracellular domain comprising a CD3 zeta signaling domain and a CD28 costimulatory molecule.

21. A pharmaceutical composition comprising the cell of claim 18, and a pharmaceutically acceptable carrier.

22. A method of generating a modified cell, the method comprising introducing into a cell the nucleic acid of claim 1.

23. The method of claim 22, wherein the cell is a regulatory T cell (Treg).

24. The method of claim 23, wherein the regulatory T cell is obtained by isolating CD4+CD25+ T cells from a population of cells.

25. The method of claim 22, wherein the regulatory T cell is obtained by modifying a T cell to express FOXP3.

26. A method of promoting α-synuclein specific immunosuppression in a subject, the method comprising administering to the subject an effective amount of a regulatory T cell comprising a CAR, wherein the CAR comprises an antigen binding domain, a transmembrane domain, and an intracellular domain, wherein the antigen binding domain specifically binds to α-synuclein.

27. A method of treating a neurodegenerative disease or a disease associated with an α-synucleinopathy in a subject, the method comprising administering to the subject an effective amount of a regulatory T cell comprising a CAR, wherein the CAR comprises an antigen binding domain, a transmembrane domain, and an intracellular domain, wherein the antigen binding domain specifically binds to α-synuclein.

28. The method of claim 27, wherein the antigen binding domain comprises a heavy chain variable (VH) domain comprising HCDR3, HCDR2, and HCDR1 regions, and a light chain variable (VL) domain comprising LCDR3, LCDR2 and LCDR1 regions, wherein
the HCDR3 region comprises the amino acid sequence AAEAY (SEQ ID NO: 5) and/or;
the HCDR2 region comprises the amino acid sequence IDPENDNT (SEQ ID NO: 6) and/or;
the HCDR1 region comprises the amino acid sequence GLNIKDYY (SEQ ID NO: 7) and/or;
the LCDR3 region comprises the amino acid sequence QHSWEIWT (SEQ ID NO: 8) and/or;
the LCDR2 region comprises the amino acid sequence YAS and/or;
the LCDR1 region comprises the amino acid sequence QSVSTSSYSY (SEQ ID NO: 10).

29. The method of claim 27, wherein the antigen binding domain comprises a heavy chain variable (VH) domain comprising an amino acid sequence least 85%, 90%, 95%, 96%, 97%, 98%, 99%, or 100% identical to SEQ ID NO: 3, and/or a light chain variable domain (VH) domain comprising an amino acid sequence least 85%, 90%, 95%, 96%, 97%, 98%, 99%, or 100% identical to SEQ ID NO: 4.

30. The method of claim 27, wherein the antigen binding domain comprises a heavy chain variable (VH) domain encoded by a nucleic acid sequence at least 85%, 90%, 95%, 96%, 97%, 98%, 99%, or 100% identical to SEQ ID NO: 1, and/or a light chain variable domain (VH) domain encoded by a nucleic acid sequence at least 85%, 90%, 95%, 96%, 97%, 98%, 99%, or 100% identical to SEQ ID NO: 2.

31. The method of claim 27, wherein the intracellular domain comprises a signaling domain and/or an intracellular domain of a costimulatory molecule.

32. The method of claim 31, wherein the signaling domain comprises a CD3 zeta signaling domain.

33. The method of claim 31, wherein the costimulatory molecule is CD28.

34. The method of claim 31, wherein the intracellular domain comprises a CD3 zeta signaling domain and a CD28 costimulatory molecule.

35. The method of claim 27, wherein the CAR comprises an amino acid sequence at least 85%, 90%, 95%, 96%, 97%, 98%, 99%, or 100% identical to SEQ ID NO: 12 or SEQ ID NO: 14.

36. The method of claim 27, wherein the CAR is encoded by a nucleic acid sequence at least 85%, 90%, 95%, 96%, 97%, 98%, 99%, or 100% identical to SEQ ID NO: 11 or SEQ ID NO: 13.

37. The method of claim 27, wherein the subject is human.

38. The method of claim 27, wherein the disease is selected from Parkinson's disease (PD), dementia with Lewy bodies (DLB), multiple system atrophy (MSA), and Alzheimer's disease with mixed Lewy pathology.

39. The method of claim 38, wherein the disease is Parkinson's disease (PD).
